(19) United States
(12) Patent Application Publication
(10) Pub. No.: US 2026/0283239 A1
(43) Pub. Date: Sep. 24, 2026

(54) SMOKING SUBSTITUTE APPARATUS

(71) Applicant: Imperial Tobacco Limited, Bristol (GB)

(72) Inventors: Samantha Murray, Bristol (GB); Chris Lord, Bristol (GB); David Jones, Bristol (GB); Jonathan Marchbank, Bristol (GB); Tom Sudlow, Bristol (GB); Ian Stuart, Bristol (GB); Stephen Jones, Bristol (GB)

(21) Appl. No.: 19/532,430

(22) Filed: Feb. 6, 2026

Related U.S. Application Data
(63) Continuation of application No. 17/395,276, filed on Aug. 5, 2021, now abandoned.
Application No. 17/395,276 is a continuation of application No. PCT/EP2020/053106, filed on Feb. 7, 2020.
Application No. PCT/EP2020/053106 is a continuation of application No. PCT/EP2020/053110, filed on Feb. 7, 2020.
Application No. PCT/EP2020/053110 is a continuation of application No. PCT/EP2020/053112, filed on Feb. 7, 2020.
Application No. PCT/EP2020/053112 is a continuation of application No. PCT/EP2020/053115, filed on Feb. 7, 2020.
Application No. PCT/EP2020/053115 is a continuation of application No. PCT/EP2020/053116, filed on Feb. 7, 2020.
Application No. PCT/EP2020/053116 is a continuation of application No. PCT/EP2020/053119, filed on Feb. 7, 2020.
Application No. PCT/EP2020/053119 is a continuation of application No. PCT/EP2020/053120, filed on Feb. 7, 2020.
Application No. PCT/EP2020/053120 is a continuation of application No. PCT/EP2020/053130, filed on Feb. 7, 2020.
Application No. PCT/EP2020/053130 is a continuation of application No. PCT/EP2020/053139, filed on Feb. 7, 2020.
Application No. PCT/EP2020/053139 is a continuation of application No. PCT/EP2020/053141, filed on Feb. 7, 2020.
Application No. PCT/EP2020/053141 is a continuation of application No. PCT/EP2020/053176, filed on Feb. 7, 2020.
Application No. PCT/EP2020/053176 is a continuation of application No. PCT/EP2020/053186, filed on Feb. 7, 2020.
Application No. PCT/EP2020/053186 is a continuation of application No. PCT/EP2020/053189, filed on Feb. 7, 2020.
Application No. PCT/EP2020/053189 is a continuation of application No. PCT/EP2020/053190, filed on Feb. 7, 2020.
Application No. PCT/EP2020/053190 is a continuation of application No. PCT/EP2020/053191, filed on Feb. 7, 2020.
Application No. PCT/EP2020/053191 is a continuation of application No. PCT/EP2020/053193, filed on Feb. 7, 2020.
Application No. PCT/EP2020/053193 is a continuation of application No. PCT/EP2020/053194, filed on Feb. 7, 2020.
Application No. PCT/EP2020/053194 is a continuation of application No. PCT/EP2020/053195, filed on Feb. 7, 2020.
Application No. PCT/EP2020/053195 is a continuation of application No. PCT/EP2020/053200, filed on Feb. 7, 2020.

(30) Foreign Application Priority Data
Feb. 7, 2019 (EP) 19155858.4
Feb. 7, 2019 (EP) 19155861.8
Feb. 7, 2019 (EP) 19155862.6
Feb. 7, 2019 (EP) 19155864.2
Feb. 7, 2019 (EP) 19155866.7
Feb. 7, 2019 (EP) 19155871.7
Feb. 7, 2019 (EP) 19155873.3
Feb. 7, 2019 (EP) 19155875.8
Feb. 7, 2019 (EP) 19155877.4
Feb. 7, 2019 (EP) 19155878.2
Feb. 7, 2019 (EP) 19155879.0
Feb. 7, 2019 (EP) 19155887.3
Feb. 7, 2019 (EP) 19155894.9
Feb. 7, 2019 (EP) 19155903.8
Feb. 7, 2019 (EP) 19155908.7
Feb. 7, 2019 (EP) 19155929.3
Feb. 7, 2019 (EP) 19155933.5
Feb. 7, 2019 (EP) 19155934.3
Feb. 7, 2019 (EP) 19155936.8
Feb. 7, 2019 (EP) 19155945.9

(51) Int. Cl. A24F 40/42; A24B 15/167; A24B 15/28; A24F 40/10; A24F 40/485; A24F 40/70; A61M 15/00

(52) U.S. Cl. CPC A24F 40/42; A24B 15/167; A24B 15/285; A24F 40/10; A24F 40/485; A24F 40/70; A61M 15/009; A61M 2205/0238

(57) ABSTRACT

A smoking substitute apparatus has a flavourant-containing element formed by printing a flavourant onto an outer surface of the smoking substitute apparatus. Alternatively a smoking substitute apparatus has a flavourant-containing element on an outer surface thereof, the flavourant-containing element comprising a scented and/or flavoured ink.

### CROSS REFERENCE TO RELATED APPLICATIONS/INCORPORATION BY REFERENCE STATEMENT

This application is a continuation of U.S. nonprovisional utility patent application Ser. No. 17/395,276, filed on 5 Aug. 2021 and published on 27 Jan. 2022 as United States patent application publication no. US 2022/0022537A1, which is a continuation of the following international patent applications: no. PCT/EP2020/053119, filed on 7 Feb. 2020, which claims priority to EP 19155894.9, filed on 7 Feb. 2019; no. PCT/EP2020/053193, filed on 7 Feb. 2020, which claims priority to EP 19155934.3, filed on 7 Feb. 2019; no. PCT/EP2020/053195, filed on 7 Feb. 2020, which claims priority to EP 19155936.8, filed on 7 Feb. 2019; no. PCT/EP2020/053112, filed on 7 Feb. 2020, which claims priority to EP 19155866.7, filed on 7 Feb. 2019; no. PCT/EP2020/053186, filed on 7 Feb. 2020, which claims priority to EP 19155903.8, filed on 7 Feb. 2019; no. PCT/EP2020/053116, filed on 7 Feb. 2020, which claims priority to EP 19155871.7, filed on 7 Feb. 2019; no. PCT/EP2020/053194, filed on 7 Feb. 2020, which claims priority to EP 19155945.9, filed on 7 Feb. 2019; no. PCT/EP2020/053189, filed on 7 Feb. 2020, which claims priority to EP 19155908.7, filed on 7 Feb. 2019; no. PCT/EP2020/053120, filed on 7 Feb. 2020, which claims priority to EP 19155873.3, filed on 7 Feb. 2019; no. PCT/EP2020/053115, filed on 7 Feb. 2020, which claims priority to EP 19155861.8, filed on 7 Feb. 2019; no. PCT/EP2020/053130, filed on 7 Feb. 2020, which claims priority to EP 19155875.8, filed on 7 Feb. 2019; no. PCT/EP2020/053139, filed on 7 Feb. 2020, which claims priority to EP 19155877.4, filed on 7 Feb. 2019; no. PCT/EP2020/053141, filed on 7 Feb. 2020, which claims priority to EP 19155878.2, filed on 7 Feb. 2019; no. PCT/EP2020/053191, filed on 7 Feb. 2020, which claims priority to EP 19155929.3, filed on 7 Feb. 2019; no. PCT/EP2020/053110, filed on 7 Feb. 2020, which claims priority to EP 19155862.6, filed on 7 Feb. 2019; no. PCT/EP2020/053106, filed on 7 Feb. 2020, which claims priority to EP 19155864.2, filed on 7 Feb. 2019; no.

PCT/EP2020/053190, filed on 7 Feb. 2020, which claims priority to EP 19155887.3, filed on 7 Feb. 2019; no. PCT/EP2020/053176, filed on 7 Feb. 2020, which claims priority to EP 19155879.0, filed on 7 Feb. 2019; and no. PCT/EP2020/053200, filed on 7 Feb. 2020, which claims priority to EP 19155858.4, filed on 7 Feb. 2019, and to EP 19155933.5, filed on 7 Feb. 2019. The entire contents of each of the above-referenced applications and the above-referenced published patent application are hereby incorporated herein by reference in their entirety.

### FIELD OF THE DISCLOSURE

The present invention relates to a smoking substitute apparatus and, in particular, a smoking substitute apparatus that is able to deliver flavor to a user.

### BACKGROUND TO THE INVENTION

The smoking of tobacco is generally considered to expose a smoker to potentially harmful substances. It is generally thought that a significant amount of the potentially harmful substances is generated through the heat caused by the burning and/or combustion of the tobacco and the constituents of the burnt tobacco in the tobacco smoke itself.

Combustion of organic material such as tobacco is known to produce tar and other potentially harmful by-products. There have been proposed various smoking substitute systems in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or a flavorant without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories. There are a number of different categories of smoking substitute systems, each utilizing a different smoking substitute approach.

One approach is the so-called “vaping” approach, in which a vaporizable liquid, typically referred to (and referred to herein) as “e-liquid”, is heated by a heating device (referred to herein as an electronic cigarette or “e-cigarette” device) to produce an aerosol vapor which is inhaled by a user. The e-liquid typically includes a base liquid as well as nicotine and/or a flavorant. The resulting vapor therefore also typically contains nicotine and/or a flavorant. The base liquid may include propylene glycol and/or vegetable glycerin.

A typical e-cigarette device includes a mouthpiece, a power source (typically a battery), a tank for containing e-liquid, as well as a heating device. In use, electrical energy is supplied from the power source to the heating device, which heats the e-liquid to produce an aerosol (or “vapor”) which is inhaled by a user through the mouthpiece.

E-cigarettes can be configured in a variety of ways. For example, there are “closed system” vaping smoking substitute systems, which typically have a sealed tank and heating element. The tank is pre-filled with e-liquid and is not intended to be refilled by an end user. One subset of closed system vaping smoking substitute systems includes a main body which includes the power source, wherein the main body is configured to be physically and electrically coupled to a consumable including the tank and the heating element. In this way, when the tank of a consumable has been emptied, that consumable is disposed of. The main body can be reused by connecting it to a new, replacement, consumable. Another subset of closed system vaping smoking substitute systems is completely disposable, and intended for one-use only.

There are also “open system” vaping smoking substitute systems which typically have a tank that is configured to be refilled by a user. In this way the entire device can be used multiple times.

An example vaping smoking substitute system is the myblu™ e-cigarette. The myblu™ e-cigarette is a closed system which includes a main body and a consumable. The main body and consumable are physically and electrically coupled together by pushing the consumable into the main body. The main body includes a rechargeable battery. The consumable includes a mouthpiece, a sealed tank which contains e-liquid, as well as a heater, which for this device is a heating filament coiled around a portion of a wick. The wick is partially immersed in the e-liquid, and conveys e-liquid from the tank to the heating filament. The device is activated when a microprocessor on board the main body detects a user inhaling through the mouthpiece. When the device is activated, electrical energy is supplied from the power source to the heating device, which heats e-liquid from the tank to produce a vapor which is inhaled by a user through the mouthpiece.

For a smoking substitute device it is desirable to deliver nicotine into the user's lungs, where it can be absorbed into the bloodstream. As explained above, in the so-called “vaping” approach, e-liquid is heated by a heating device to produce an aerosol vapor which is inhaled by a user. Many e-cigarettes also deliver flavor to the user to enhance the experience. In such e-cigarettes, flavor compounds are contained in the e-liquid that is heated. However, toxicology restrictions are placed on the amount of flavor that can be contained in the e-liquid, and this can result in some e-liquid flavors delivering a weak and underwhelming taste sensation to consumers in the pursuit of safety. Further, there is a view that providing a flavorant as part of the e-liquid, such that the flavorant is vaporized with the e-liquid, may be disadvantageous.

An alternative to the “vaping” approach is the so-called Heated Tobacco (“HT”) approach in which tobacco (rather than an e-liquid) is heated or warmed to release vapor. HT is also known as “heat not burn” (“HNB”). The tobacco may be leaf tobacco or reconstituted tobacco. In the HT approach the intention is that the tobacco is heated but not burned, i.e., the tobacco does not undergo combustion.

The heating, as opposed to burning, of the tobacco material is believed to cause fewer, or smaller quantities, of the more harmful compounds ordinarily produced during smoking. Consequently, the HT approach may reduce the odor and/or health risks that can arise through the burning, combustion and pyrolytic degradation of tobacco.

A typical HT smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes components in the tobacco material to be released as vapor. A vapor may also be formed from a carrier in the tobacco material (this carrier may for example include propylene glycol and/or vegetable glycerin) and additionally volatile compounds released from the tobacco. The released vapor may be entrained in the airflow drawn through the tobacco.

As the vapor passes through the consumable (entrained in the airflow) from the location of vaporization to an outlet of the consumable (e.g., a mouthpiece), the vapor cools and condenses to form an aerosol for inhalation by the user. The aerosol may contain nicotine and/or flavor compounds.

There may be a need for improved design of smoking substitute systems, in particular in regards to the delivery of flavor to a user.

The present disclosure has been devised in the light of the above considerations.

### SUMMARY OF THE INVENTION

When there is no flavorant present in the e-liquid, or an amount of flavorant is present which delivers a weak and underwhelming taste sensation to consumers, it is desirable to supplement this with flavorant which is located elsewhere on or in the smoking substitute apparatus. Broadly speaking, the present application relates to inventions in which a smoking substitute apparatus is provided with a flavorant somewhere on its outside surface, such that when a consumer puffs on the smoking substitute device, they are also able to smell and/or taste the flavorant, to provide them with a more pleasurable “smoking” experience. This application also relates to kits of parts including components allowing a user to apply a flavorant of choice to the outside surface of their smoking substitute apparatus, in order to achieve the same effect.

A first aspect of the present invention provides a smoking substitute apparatus having a flavorant-containing element printed onto an outer surface thereof. In some embodiments of the first aspect of the invention the flavorant-containing element may be colorless.

However, in preferred embodiments of the first aspect of the invention, the flavorant containing element may comprise a flavored and/or scented ink. In embodiments in which the flavorant-containing element contains ink, the ease with which a user may locate the flavorant-containing element is improved. The flavorant-containing element may be printed onto the outer surface of the smoking substitute apparatus using pad printing or tampo printing, a method in which a 2D image is transferred to a 3D surface.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable. Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Throughout this application, the term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances. The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations. The flavorant may be configured to release aroma or flavor in response to one or more of heat, pressure, friction and moisture.

Where the smoking substitute apparatus is in the form of a consumable, the consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

A second aspect of the present invention provides a smoking substitute apparatus having a flavorant-containing element on its outer surface, the flavorant-containing element comprising a scented and/or flavored ink. Like the first aspect of the present invention, the scented and/or flavored ink of the second aspect of the invention may be applied to the smoking substitute apparatus by printing, e.g., by pad printing or tampo printing, as discussed above. Alternatively, the flavorant-containing element may be in the form of a sticker which is applied to the smoking substitute apparatus. The flavorant-containing element may be applied by spraying. The flavorant-containing element may be applied in a process wherein the regions of the smoking substitute apparatus to be coated are given a negative charge, and the ink is given a positive charge which is then attracted to the negatively charged regions of the smoking substitute apparatus to form the flavorant containing element. As will be appreciated, the charges in such an embodiment may be reversed. In an alternative embodiment, the flavorant-containing element may be applied to the inner surface of a mold which is used to form at least a portion of the smoking substitute apparatus by injection molding, such that when a heated precursor composition is injected into the mold, the flavorant-containing element is picked up by the heated precursor composition and is thereby affixed to an outer surface of that portion of the smoking substitute apparatus. The skilled person appreciates that there are several other different ways in which the flavorant-containing element may be applied to the surface of the smoking substitute apparatus.

For the purposes of the present application, the “top” side/surface of a smoking substitute apparatus is defined as the side/surface which faces upwards during normal use of the smoking substitute apparatus. Generally, when the smoking substitute apparatus is in the form of a consumable, when the consumable is engaged with a main body as described above, the top side of the consumable is aligned with the top side of the main body. In embodiments of the first aspect of the invention in which the flavorant-containing element is or includes a scented element, it is preferred that the scented element be located on a top surface of the smoking substitute apparatus, enabling the user to smell the scented element as the same time as they are using the device. In embodiments in which the flavorant containing element is or includes a flavored element, it is preferred that the flavored element be located on a bottom surface of the smoking substitute apparatus, enabling the user's tongue to contact the flavored element during use, so that the taste of the flavored element is able to complement the taste of, e.g., the vaporized-liquid.

In some embodiments, the flavorant-containing element may be both flavored and scented, and optionally the smoking substitute apparatus may include a plurality of such elements (i.e., a plurality of elements, each of which is both flavored and scented). In other embodiments, the smoking substitute apparatus may separately include a flavored element and a scented element, optionally located on the bottom surface and the top surface of the smoking substitute apparatus respectively.

In some embodiments, the flavorant-containing element may include a first flavorant layer and a second flavorant layer. The first flavorant layer may be a different flavor from the second flavorant layer. In this way, as the user continues to lick the flavorant-containing element during vaping, they are able to taste a succession of different flavors. In some embodiments there may be more than two flavorant layers, each having different flavors.

In some embodiments, the flavorant-containing element may be in the form of a visual graphic. In embodiments in which the flavorant-containing element is in the form of a visual graphic, it is preferably configured to indicate visually how much flavorant is remaining in the flavorant-containing element. In this way, a user of the smoking substitute apparatus is able to identify when the flavorant has run out, and needs, e.g., to be replaced. In one such embodiment, the flavorant-containing element may be configured to fade over time, wherein the extent to which the flavorant-containing element has faded provides the visual indication of the amount of flavorant is remaining in the flavorant-containing element. In other embodiments, the visual graphic may be configured to change color as the flavorant is volatilized or consumed by the user. For example, the graphic may include an ink which is configured to change color as the flavorant component of that ink is released.

Alternatively, the visual graphic may include a flavorant-containing layer and an ink layer, the flavorant-containing layer on top of the ink layer such that as the flavorant-containing layer evaporates or is consumed by the user, the ink layer is exposed.

In some embodiments, the flavorant-containing element may comprise a thermochromic ink, or a smart polymer which is configured to change color in response to the application of heat.

### Other Features of the Smoking Substitute Apparatus

The above description relates primarily of features of the present invention which relate in some manner to the delivery of flavorant to a user. The optional features set out below are applicable to all four of the aspects of the invention which are discussed above.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable. The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The invention includes the combination of the aspects and preferred features described except where such a combination is clearly impermissible or expressly avoided.

### SUMMARY OF THE FIGURES

Aspects and embodiments of the present invention will now be discussed with reference to the accompanying drawings, in which:

FIGS. 1A to 1C show a general smoking substitute system configured to provide an e-liquid to a user.

FIG. 2A is a front view of a second embodiment of a smoking substitute system with a consumable of the system engaged with a device of the system.

FIG. 2B is a front view of the second embodiment of the smoking substitute system with the consumable of the system disengaged from the device.

FIG. 2C is a schematic of the consumable of the second embodiment of the substitute smoking system.

FIG. 3 is a drawing of a smoking substitute apparatus having a flavorant-containing element in the form of a visual graphic printed thereon.

Further aspects and embodiments will be apparent to those skilled in the art. All documents mentioned in this text are incorporated herein by reference.

### DETAILED DESCRIPTION OF THE FIGURES

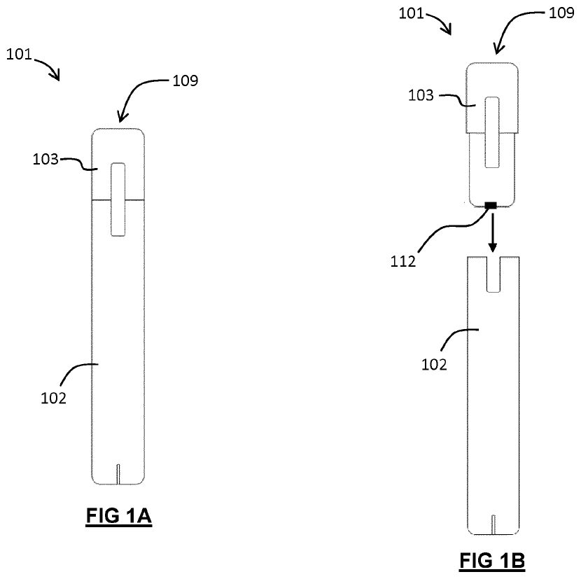

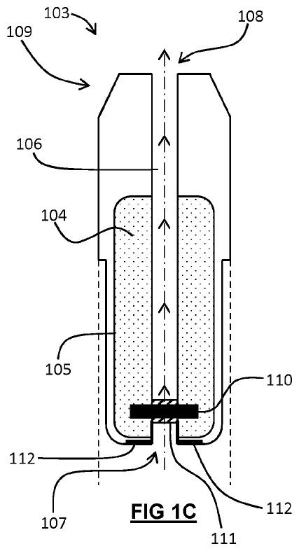

FIGS. 1A and 1B illustrate a smoking substitute system in the form of an e-cigarette system 101. The system 101 comprises an e-cigarette device defining a main body 102 of the system 101, and a smoking substitute apparatus in the form of an e cigarette consumable (or “pod”) 103. In the illustrated embodiment the consumable 103 (smoking substitute apparatus) is removable from the main body (e-cigarette device), so as to be a replaceable component of the system 101. In other words, the e-cigarette system 101 is a closed system.

As is apparent from FIGS. 1A and 1B, the consumable 103 is configured to engage the main body 102. FIG. 1A shows the main body 102 and the consumable 103 in an engaged state, whilst FIG. 1B shows the main body 102 and the consumable 103 in a disengaged state. When engaged, a portion of the consumable 103 is received in a cavity of the main body 102 and is retained in the engaged position by way of a snap-engagement mechanism. In other embodiments, the main body 102 and consumable 103 may be engaged by screwing one into (or onto) the other, through a bayonet fitting, or by way of an interference fit.

The system 101 is configured to vaporize an aerosol-former, which in the illustrated embodiment, is in the form of a nicotine-based e-liquid 104. The e-liquid 104 comprises nicotine and a base liquid including propylene glycol and/or vegetable glycerin. In the present embodiment, the e-liquid 104 is flavorless (and does not include any added flavorant). That is, if the e-liquid 104 were to be inhaled (i.e., in aerosol form) by a user, it would not have a particularly perceptible flavor or taste.

As is more apparent from FIG. 1C, this e-liquid 104 is stored within a reservoir in the form of a tank 105 that forms part of the consumable 103. In the illustrated embodiment, the consumable 103 is a “single-use” consumable 103. That is, upon exhausting the e-liquid 104 in the tank 105, the intention is that the user disposes of the entire consumable 103. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105 surrounds, and thus defines a portion of, a passage 106 that extends between an inlet 107 and an outlet 108 at opposing ends of the consumable 103. In this respect, the passage comprises an upstream end at the end of the consumable 103 that engages with the main body 102, and a downstream end at an opposing end of the consumable 103 that comprises a mouthpiece 109 of the system 101. When the consumable 103 is engaged with the main body 102, a user can inhale (i.e., take a puff) via the mouthpiece 109 so as to draw air through the passage 106, and so as to form an airflow (indicated by arrows) in a direction from the inlet 107 to the outlet 108 of the passage 106. Although not illustrated, the passage 106 may be partially defined by a tube (e.g., a metal tube) extending through the consumable 103. The passage 106 is in fluid communication with a gap defined between the consumable 103 and the main body 102 (when engaged) such that air outside of the system 101 is drawn into the passage 106 (during an inhale).

The smoking substitute system 101 is configured to vaporize the e-liquid 104 for inhalation by a user. To provide this, the consumable 103 comprises a heater having of a porous wick 110 and a resistive heating element in the form of a heating filament 111 that is helically wound around a portion of the porous wick 110. The porous wick 110 extends across the passage 106 (i.e., transverse to a longitudinal axis of the passage 106) and opposing ends of the wick 110 extend into the tank 105 (so as to be submerged in the e liquid 104). In this way, e-liquid 104 contained in the tank 105 is conveyed from the opposing ends of the porous wick 110 to a central portion of the porous wick 110 so as to be exposed to the airflow in the passage 106 (i.e., caused by a user inhaling).

The helical filament 111 is wound about this exposed central portion of the porous wick 110 and is electrically connected to an electrical interface in the form of electrical contacts 112 mounted at the end of the consumable that is proximate the main body 102 (when engaged). When the consumable 103 is engaged with the main body 102, the electrical contacts 112 contact corresponding electrical contacts (not shown) of the main body 102. The main body electrical contacts are electrically connected to a power source (not shown) of the main body 102, such that (in the engaged position) the filament 111 is electrically connected to the power source. In this way, power can be supplied by the main body 102 to the filament 111 in order to heat the filament 111. This heat is transferred from the filament 111 to the porous wick 110 which causes e-liquid 104 conveyed by the porous wick 110 to increase in temperature to a point at which it vaporizes. The vaporized e-liquid becomes entrained in the airflow and, between the vaporization point at the filament 111 and the outlet 108 of the passage 106, condenses to form an aerosol. This aerosol is then inhaled, via the mouthpiece 109, by a user of the system 101.

The power source of the main body 102 may be in the form of a battery (e.g., a rechargeable battery). The main body 102 may comprise a connector in the form of, e.g., a USB port for recharging this battery. The main body 102 may also comprise a controller that controls the supply of power from the power source to the main body electrical contacts (and thus to the filament 111). That, is the controller may be configured to control a voltage applied across the main body electrical contacts, and thus the voltage applied across the filament 111. In this way, the filament 111 may only be heated under certain conditions (e.g., during a puff and/or only when the system is in an active state). In this respect, the main body 102 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102 and consumable 103 may comprise a further interface which may, for example, be in the form of an RFID reader, a barcode or QR code reader. This interface may be able to identify a characteristic (e.g., a type) of a consumable 103 engaged with the main body 102. In this respect, the consumable 103 may include any one or more of an RFID chip, a barcode or QR code, or memory within which is an identifier and which can be interrogated via the interface.

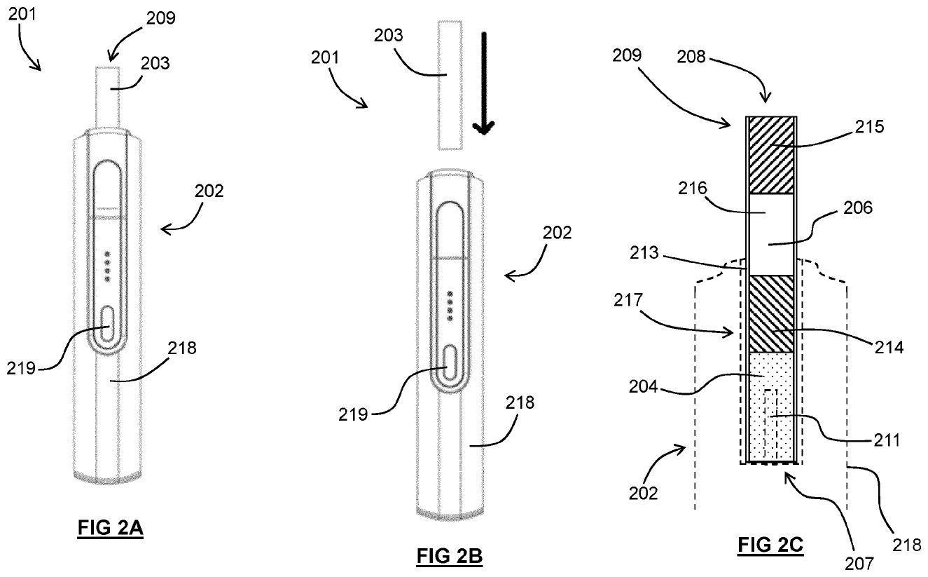

FIGS. 2A and 2B illustrate a smoking substitute system the form of a heated tobacco (HT) system 201. The system 201 comprises an HT device 202 and an aerosol-forming article in the form of a HT consumable 203.

The consumable 203 is configured to engage the device 202 by way of an interference fit. FIG. 2A shows the device 202 and the consumable 203 in an engaged state, and FIG. 2B shows the device 202 and the consumable 203 in a disengaged state.

The consumable 203 generally resembles a cigarette. In this respect, the consumable 202 has a generally cylindrical form with a diameter of approximately 7 mm and an axial length of approximately 70 mm. The consumable 203 comprises an outer wrapping layer 213 defining a passage 206 extending between openings at opposing ends of the consumable 203.

Thus, the passage comprises an upstream end (and opening) 207 at the end of the consumable 203 that engages with the device 202, and a downstream end (and opening) 208 at an opposing end of the consumable 203 that projects from the device 202 (when engaged) and defines a mouth end 209 of the system 201. When the consumable 203 is engaged with the device 202, a user can inhale (i.e., take a puff) via the mouth end 209 so as to draw air through the passage 206, and so as to form an airstream in a direction from the upstream end 207 to the downstream end 208 of the passage 206.

The consumable 203 comprises an aerosol former in the form of an aerosol-forming substrate 204 that is disposed at the upstream end 207 of the passage 206. The consumable 203 further comprises an upstream filter 214 adjacent the aerosol-forming substrate 204, a terminal filter 215 at the downstream end 208, and a spacer 216 interposed between the terminal 215 and upstream 214 filters.

The aerosol forming substrate 204 comprises tobacco material that may, for example, include any suitable parts of the tobacco plant (e.g., leaves, stems, roots, bark, seeds and flowers). In order to generate an aerosol, the aerosol forming substrate 204 comprises at least one volatile compound that is intended to be vaporized/aerosolized and that may provide the user with a recreational and/or medicinal effect when inhaled. The aerosol forming substrate 204 may further comprise one or more additives. For example, such additives may be in the form of humectants (e.g., propylene glycol and/or vegetable glycerin), flavorants, fillers, aqueous/non-aqueous solvents and/or binders.

The device comprises a heating element 211 that projects into the aerosol-forming substrate 204 when the consumable 203 is engaged with the device 202. This heating element 211 is electrically connected to a power supply (not shown) of the device 202 and, when activated, heats the aerosol-forming substrate 204 such that vapor is released from the aerosol forming substrate 204. When a user inhales via the mouth end 209, air is drawn through the heated aerosol-forming substrate 204 and the vapor becomes entrained in the resultant airflow. As the vapor flows from the aerosol-forming substrate 204 to the downstream end 208 of the passage 206 (through the filters 214, 215 and the spacer 216), it condenses into an aerosol and the aerosol is inhaled by the user.

As is apparent from FIG. 2C, when engaged, the consumable 203 is received in a cavity 217 formed in a housing 218 of the device 202. Whilst not shown in the figures, the housing 218 accommodates (in addition to the abovementioned power supply) a controller for controlling power supply to the heating element 211. Control of the heating element 211 may be performed in response to a user input (e.g., via a button 219 disposed on the housing 218) and/or a signal received from a puff sensor (configured to indicate a puff state).

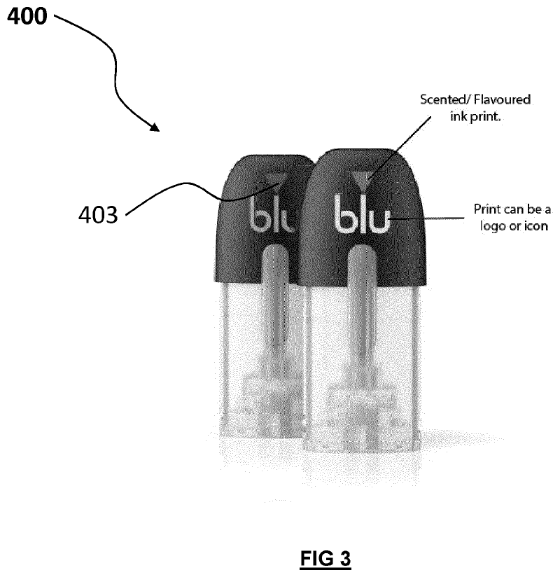

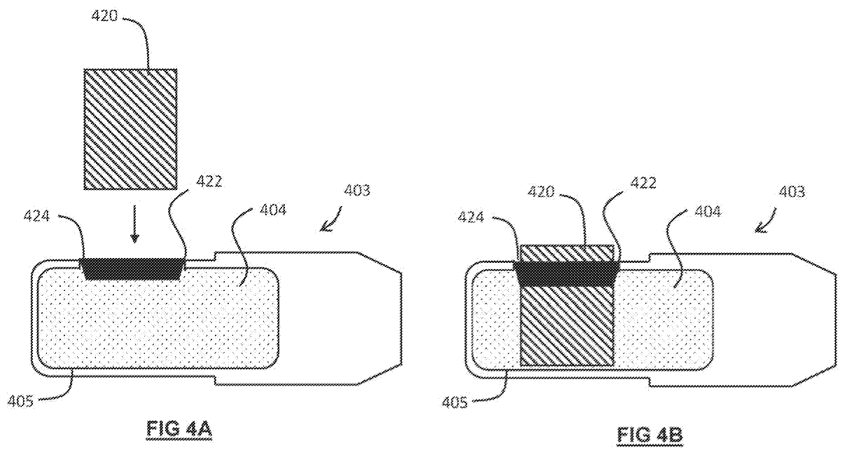

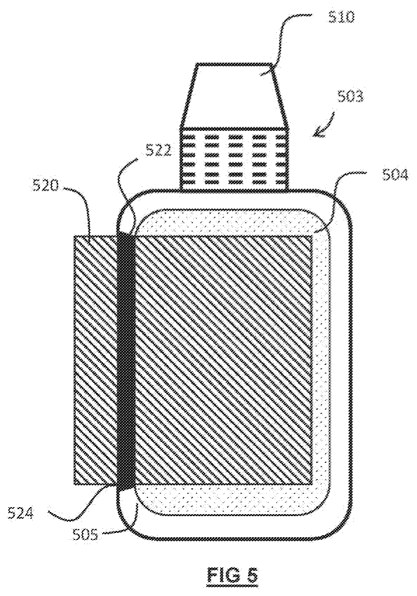

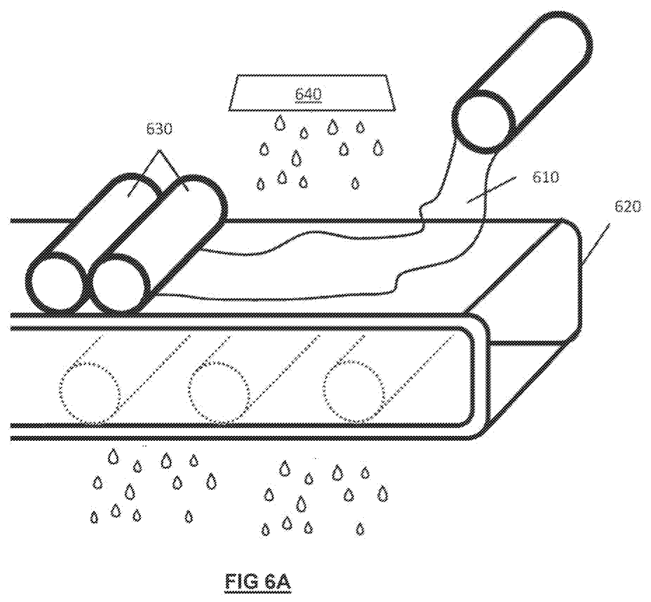

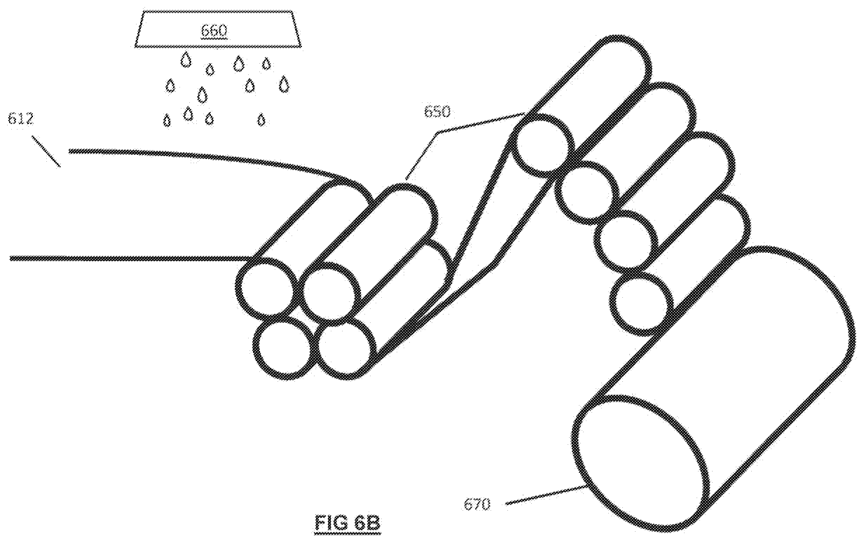

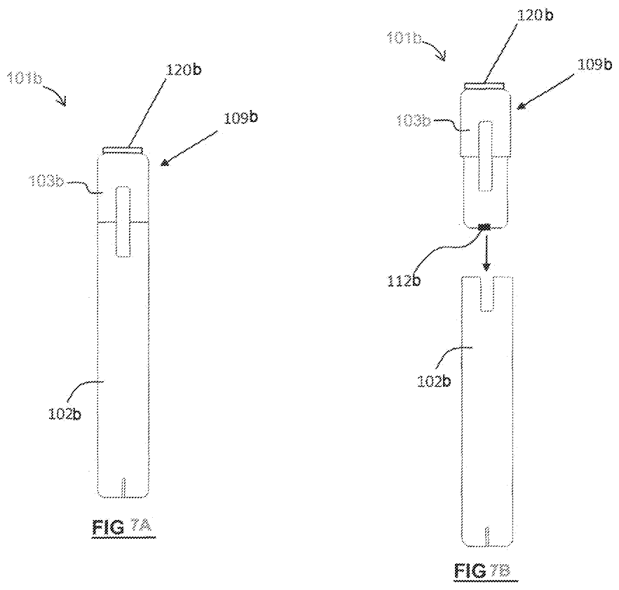

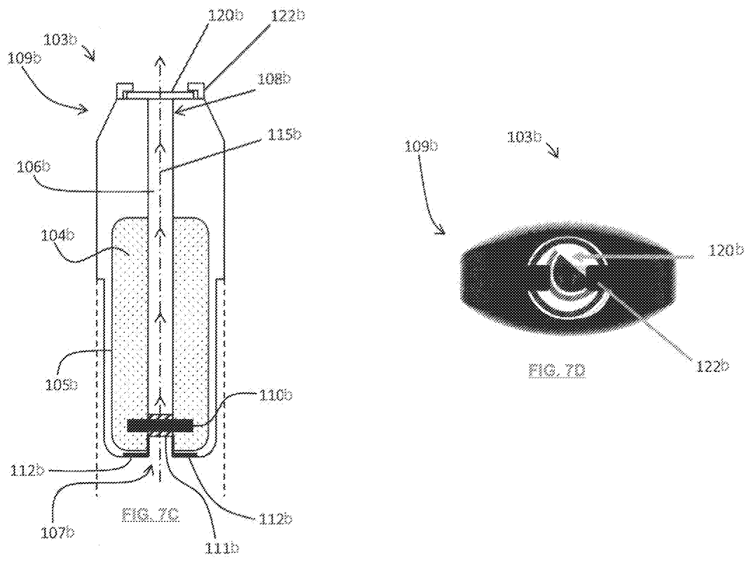

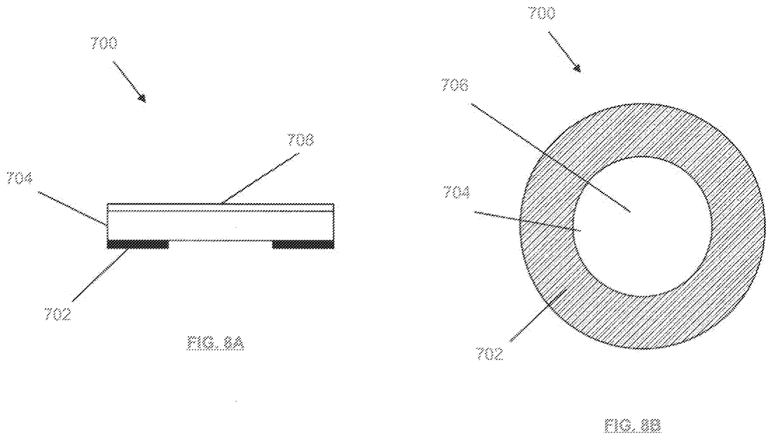

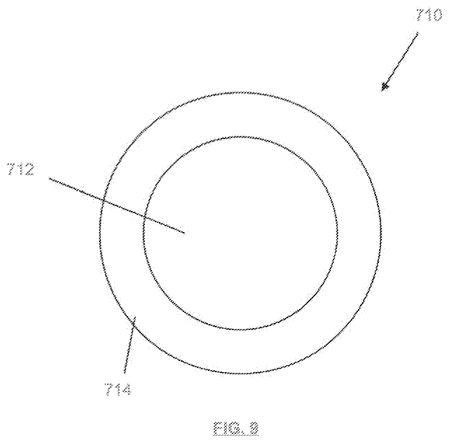

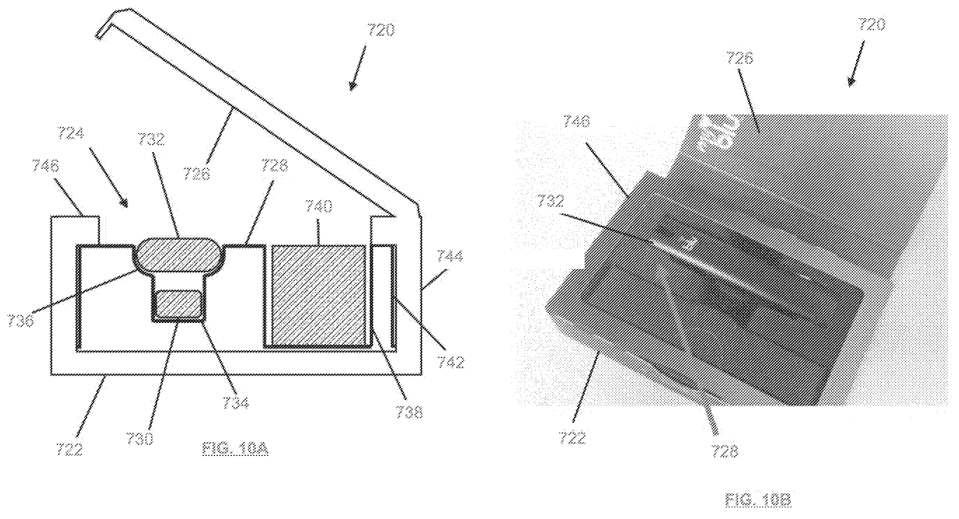

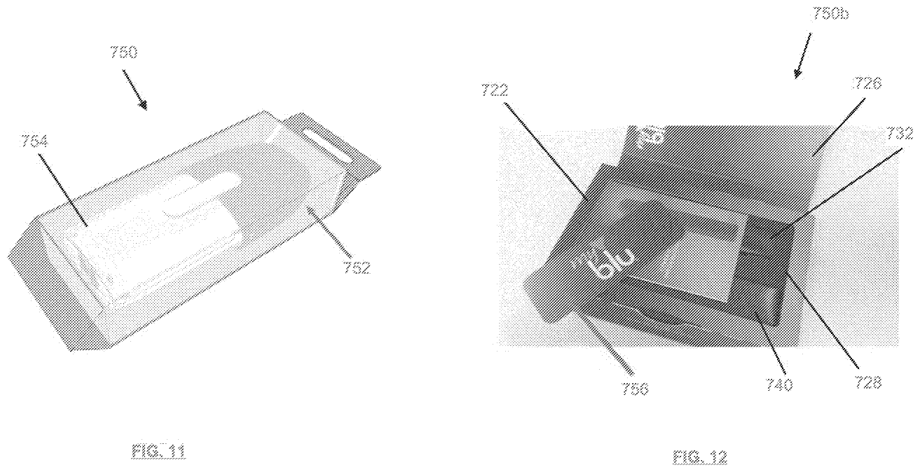

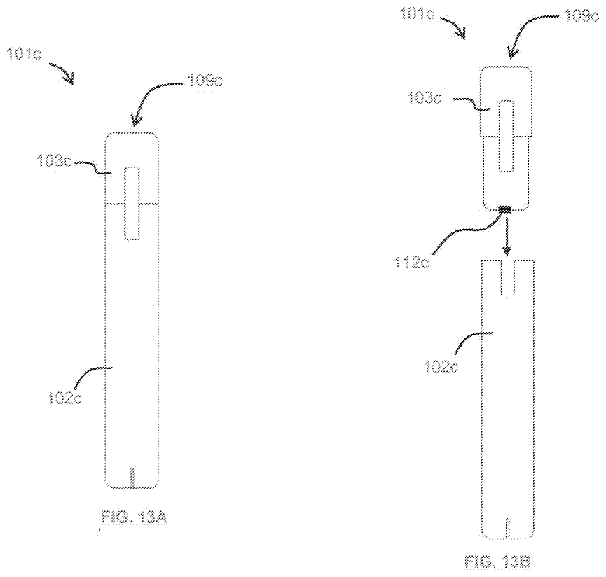

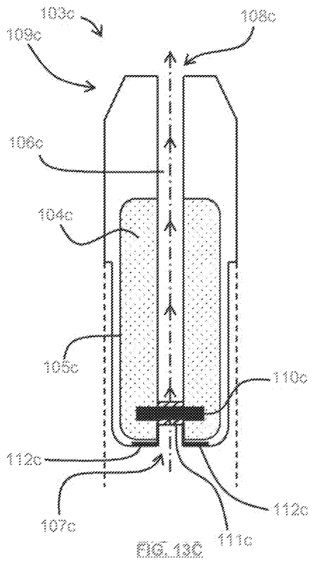

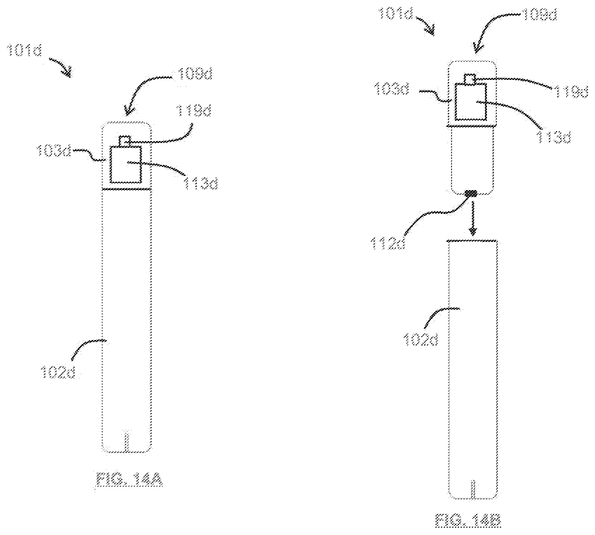

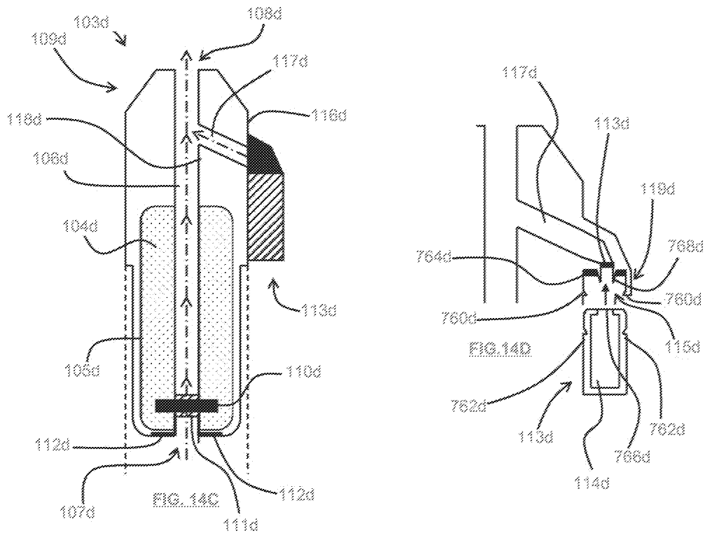

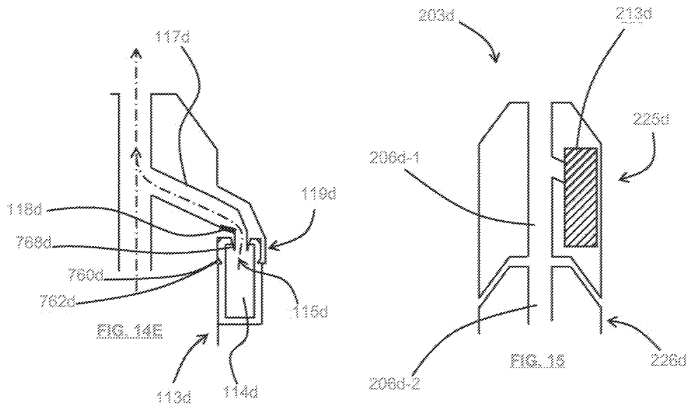

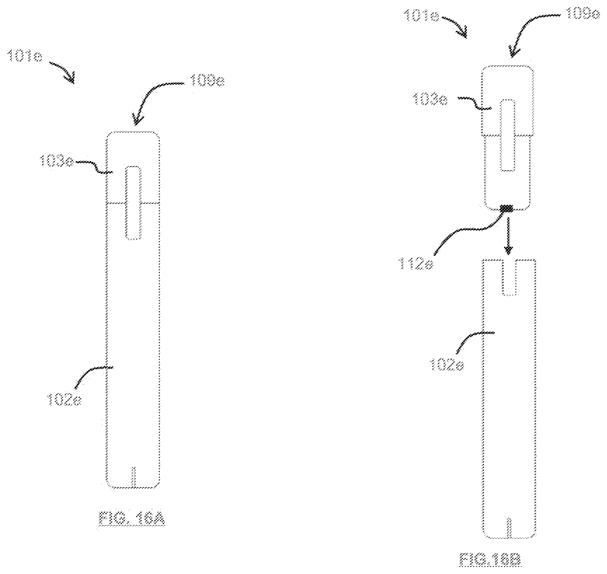

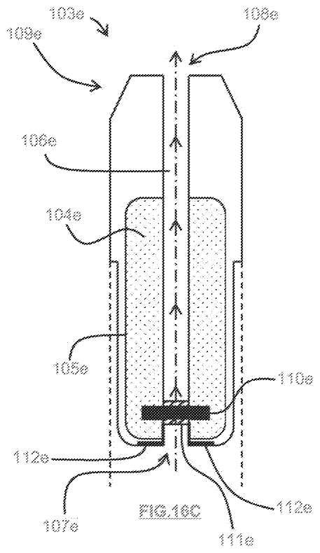

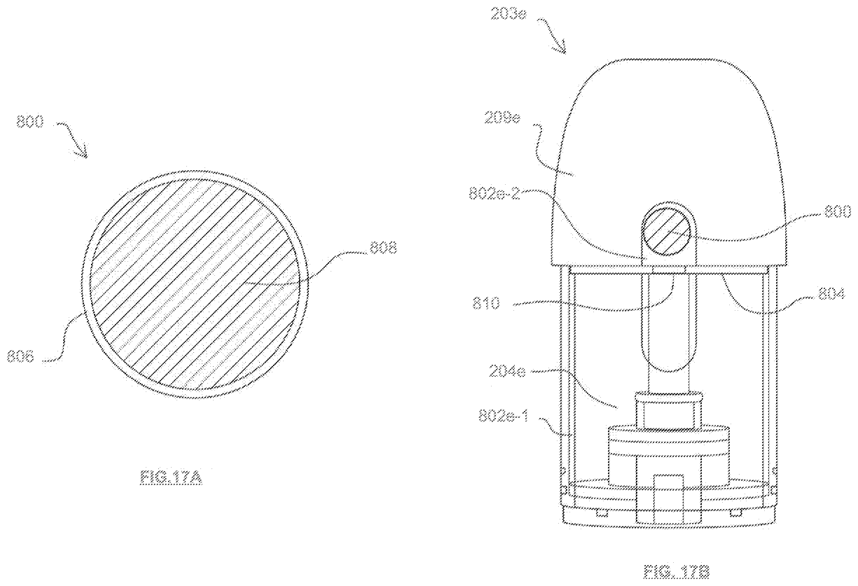

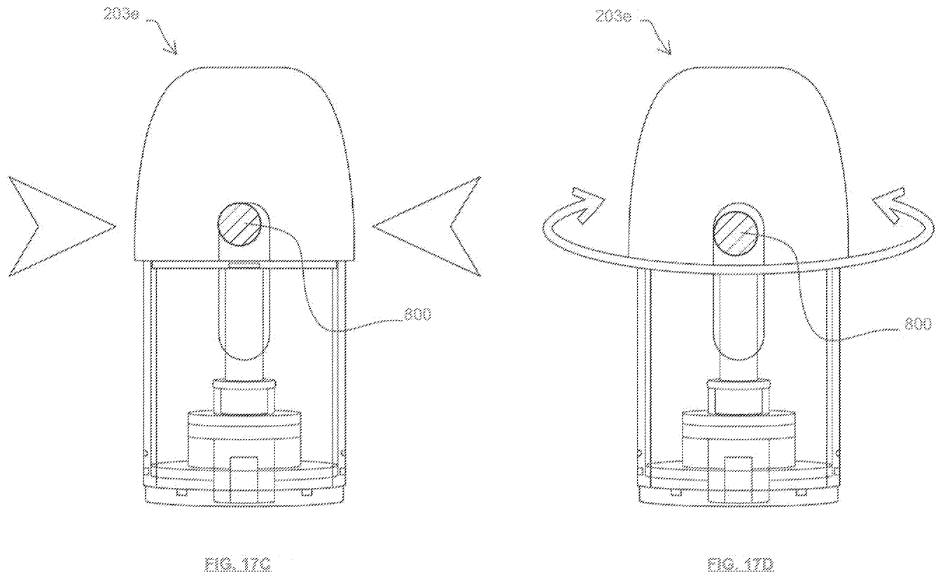

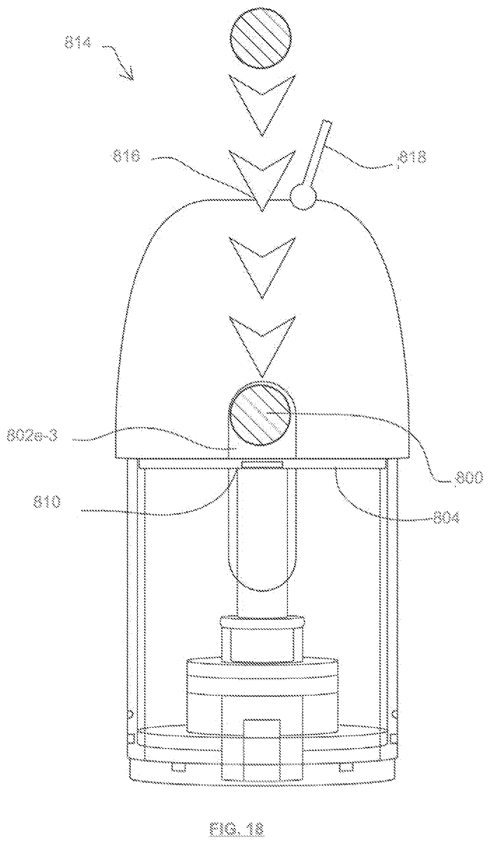

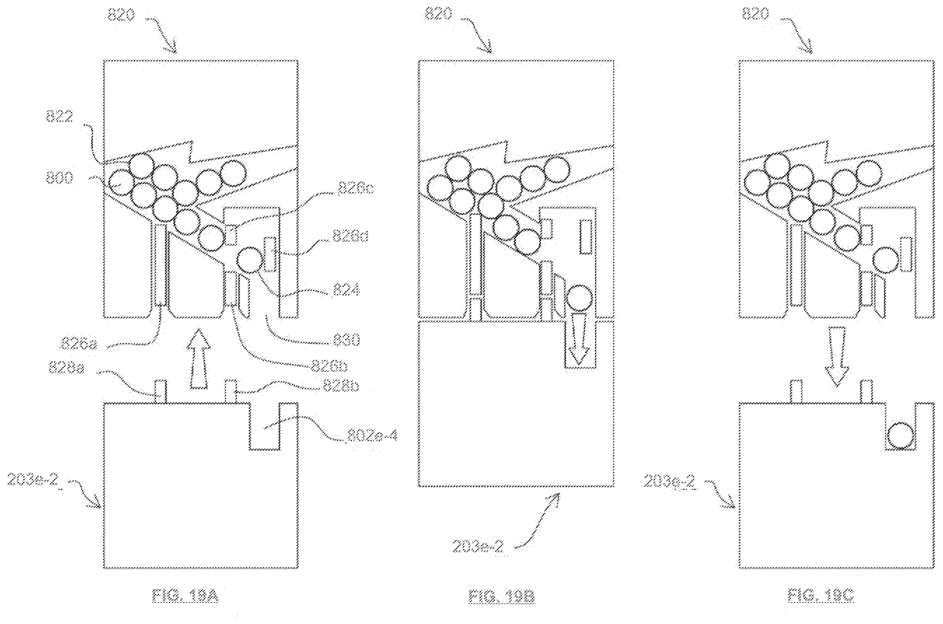

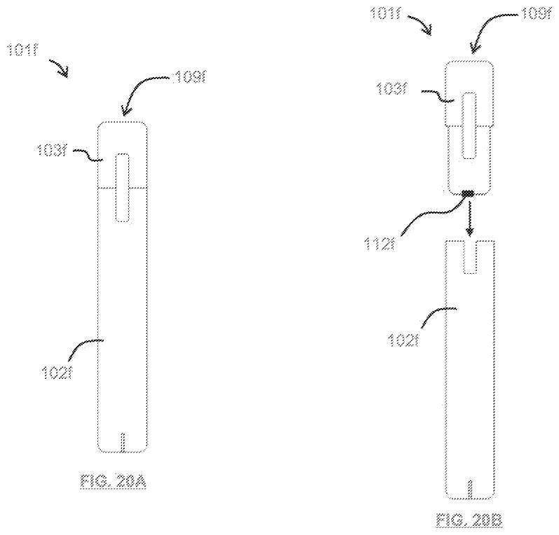

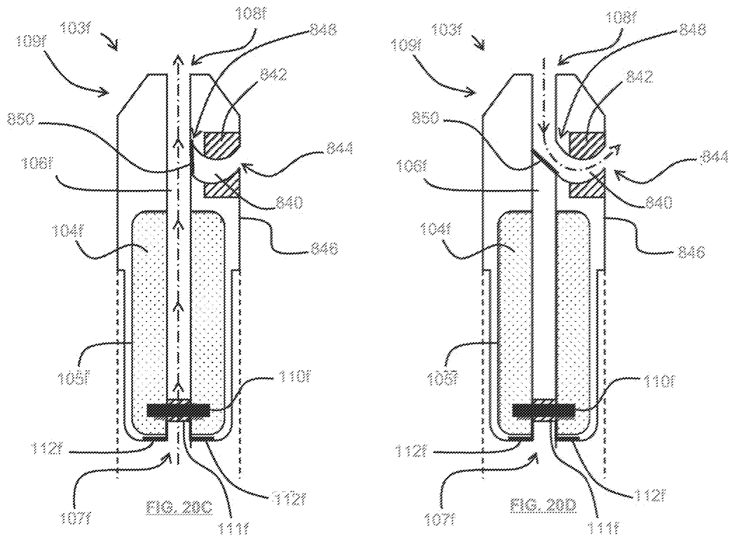

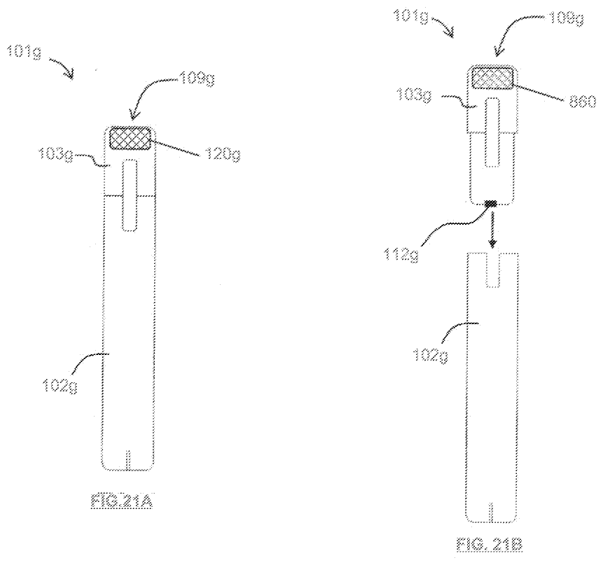

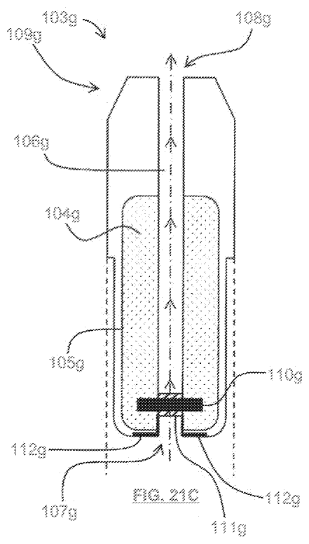

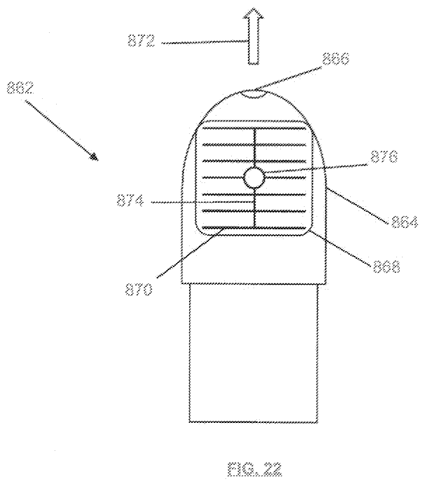

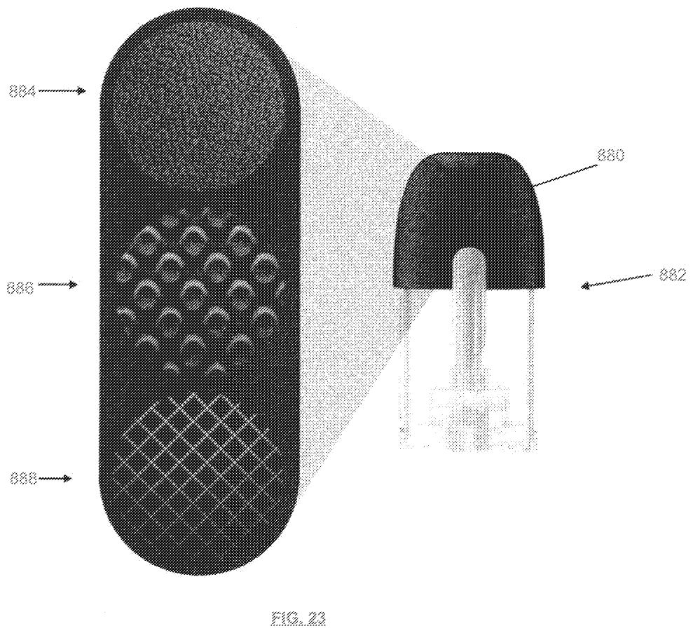

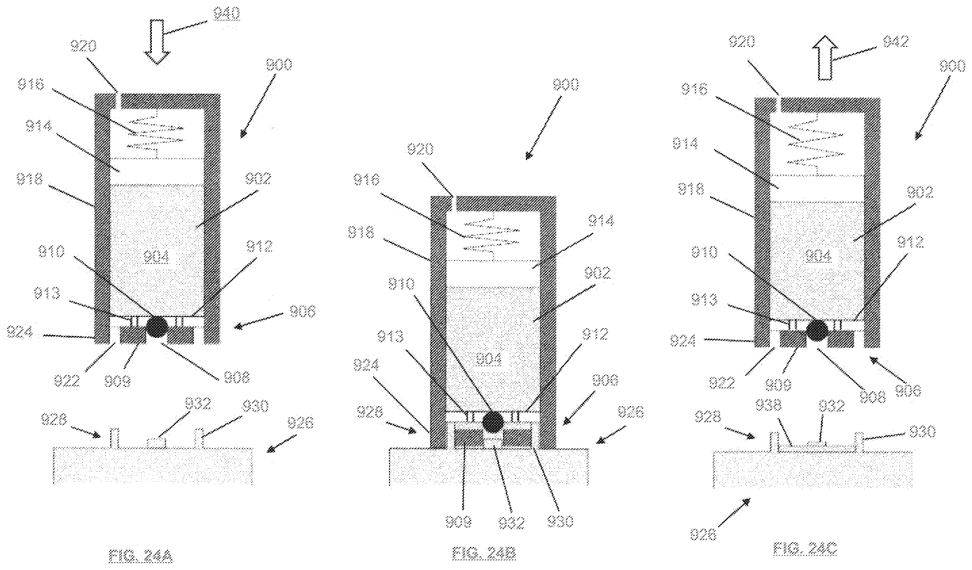

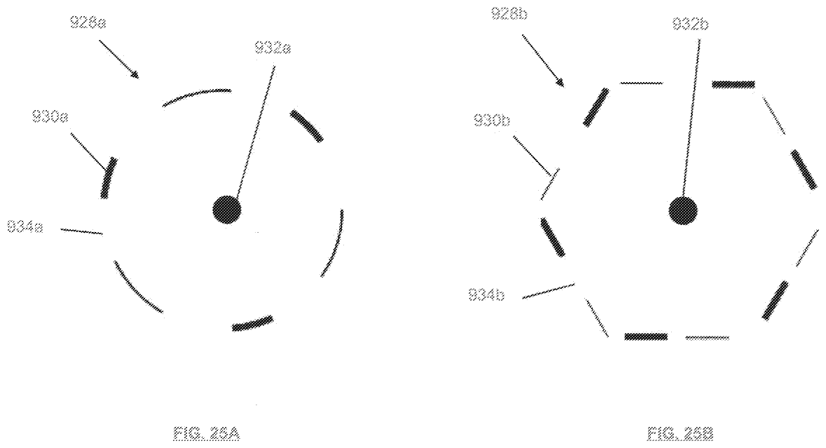

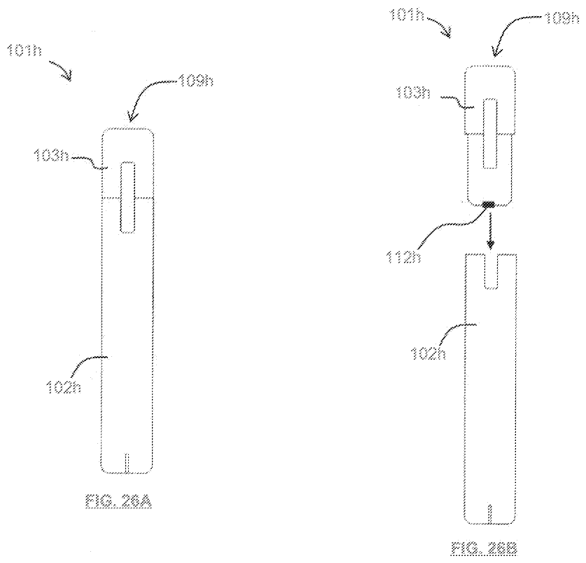

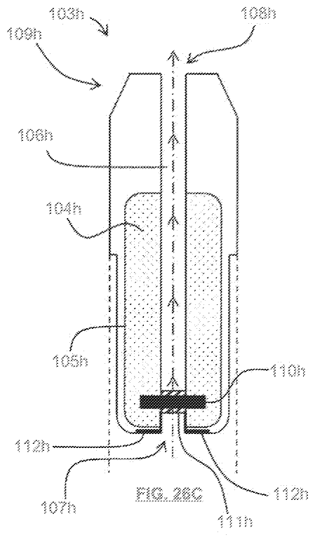

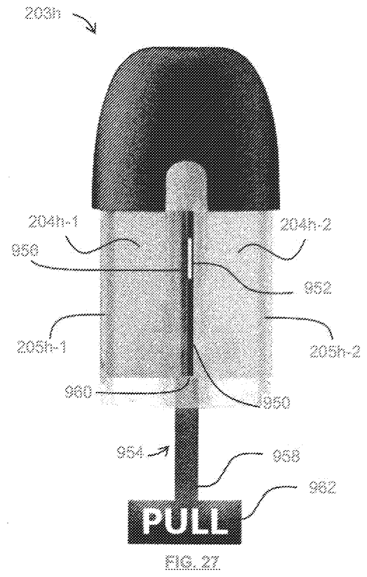

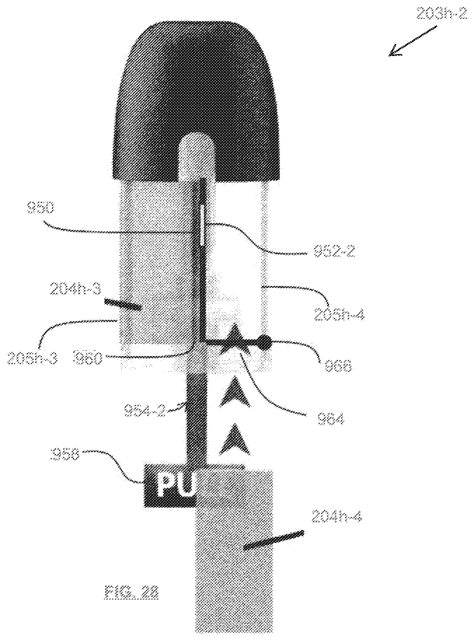

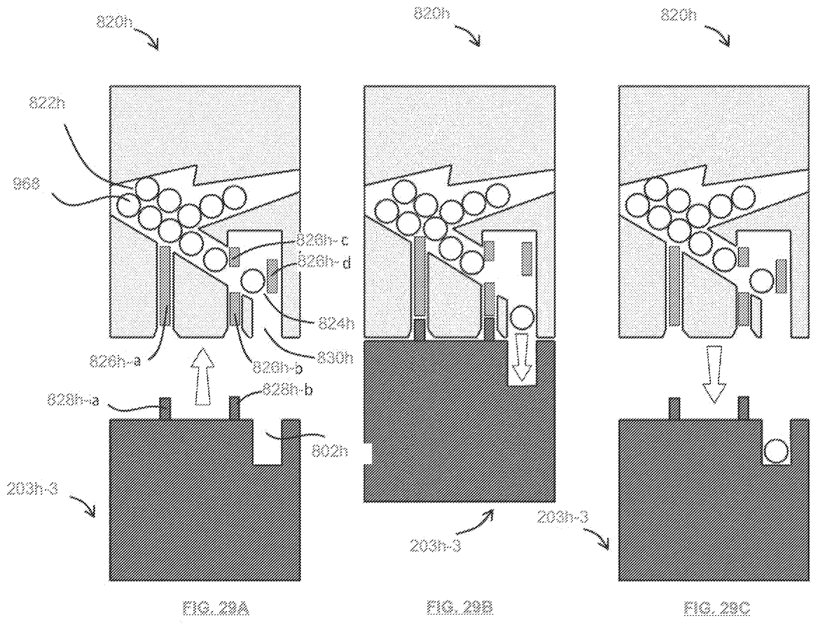

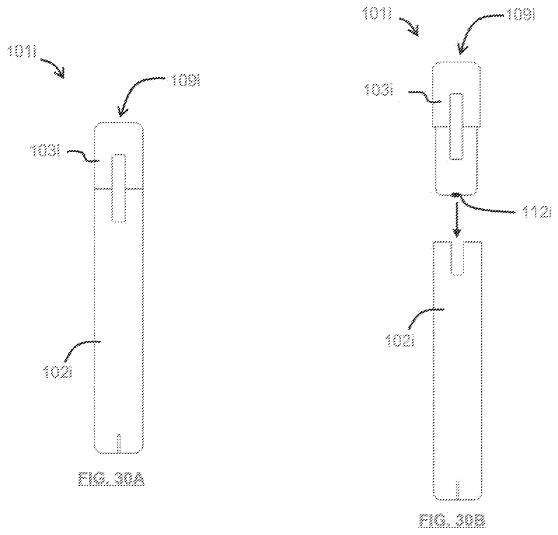

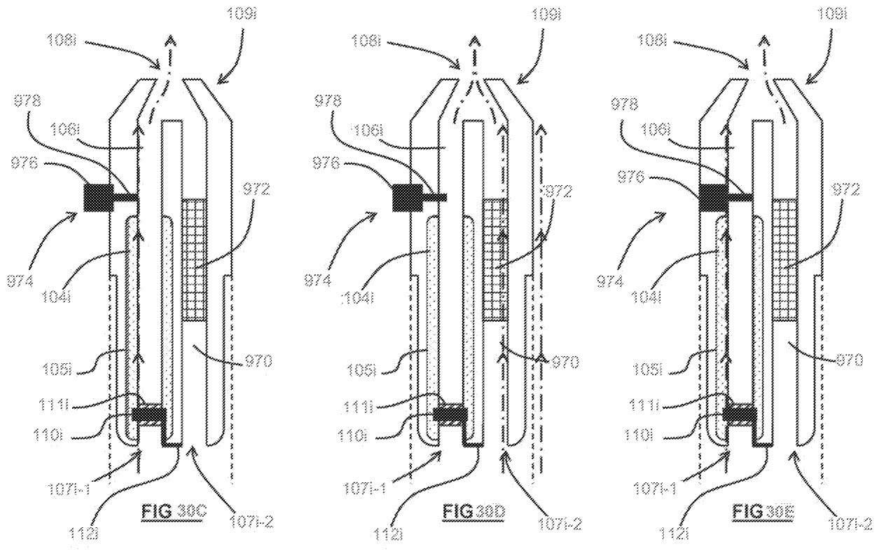

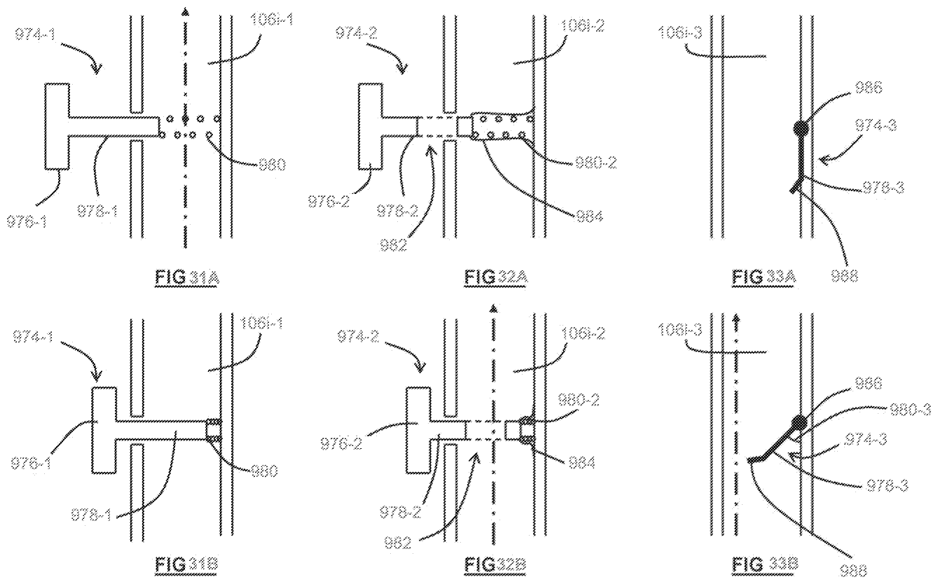

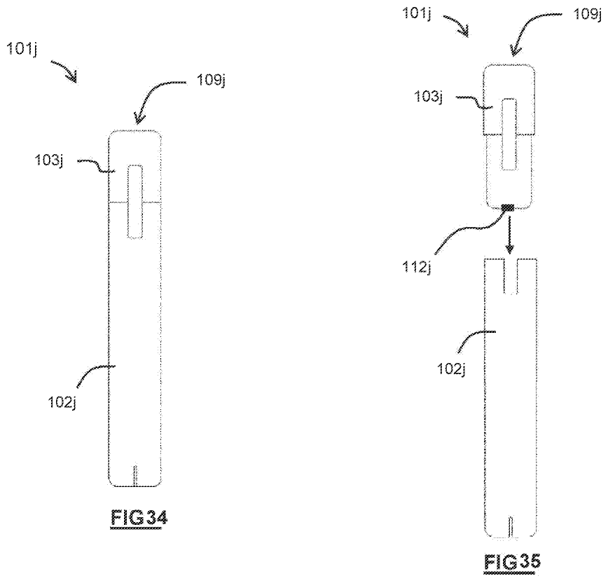

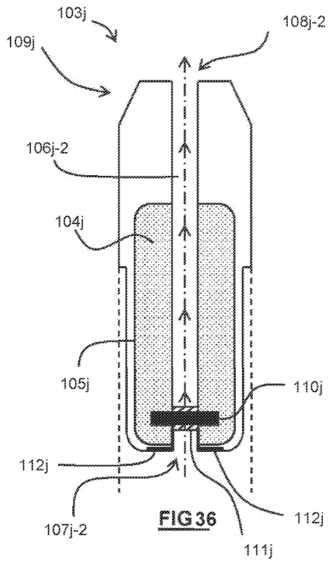

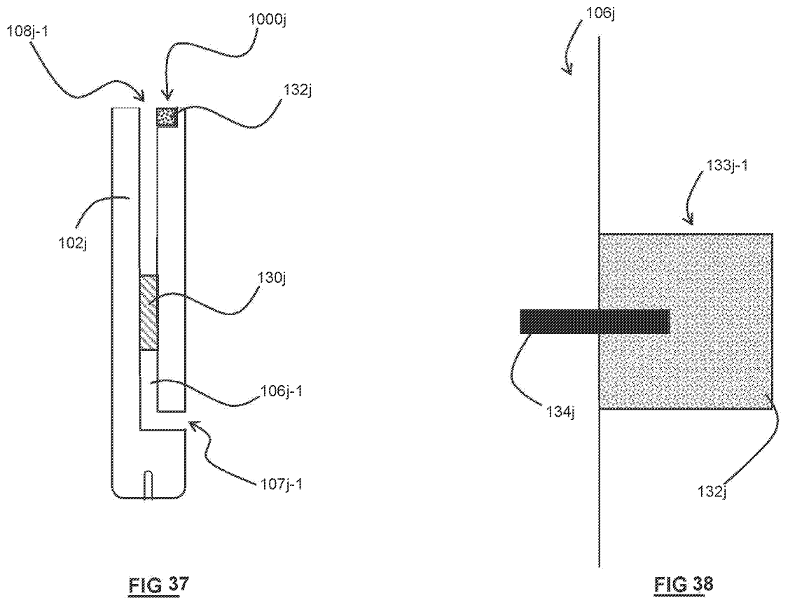

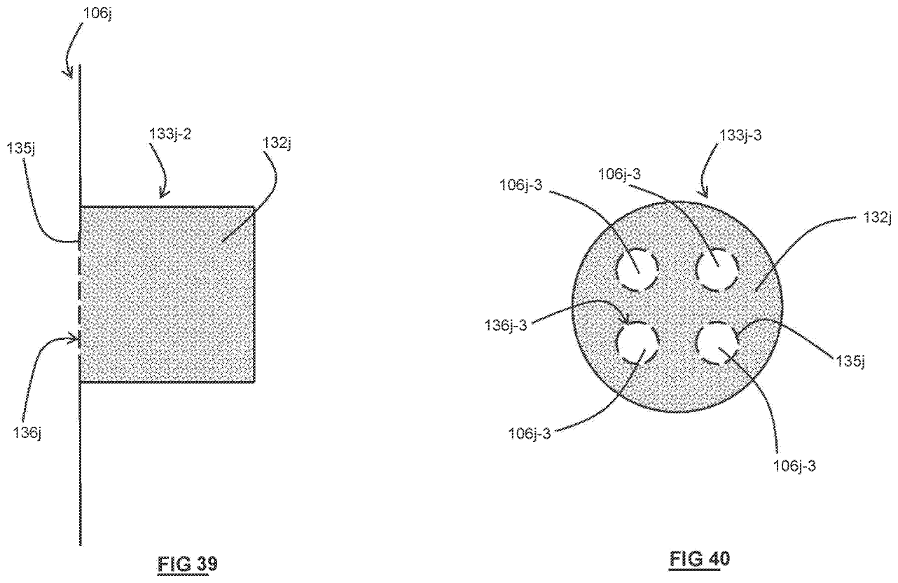

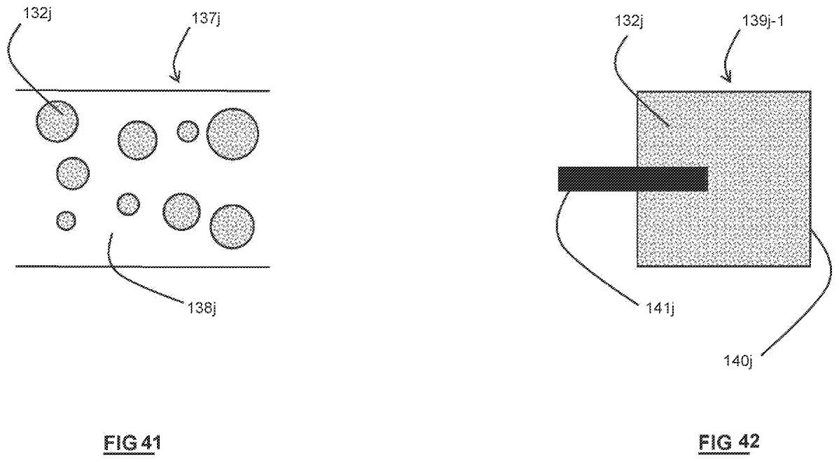

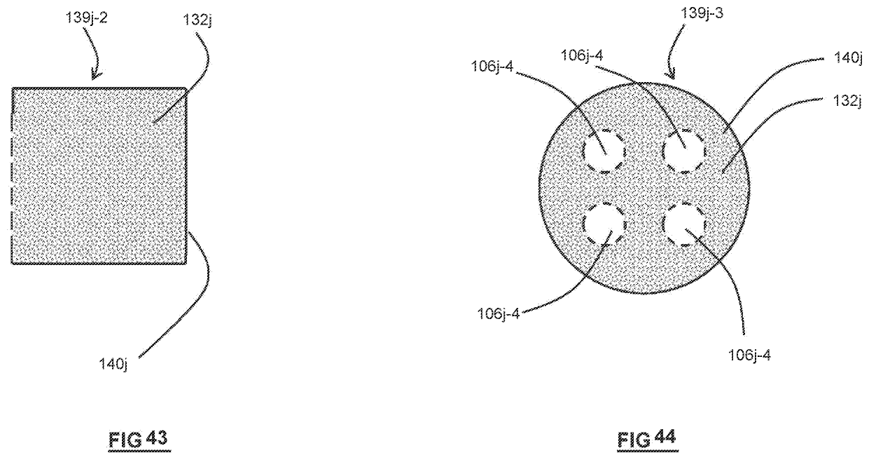

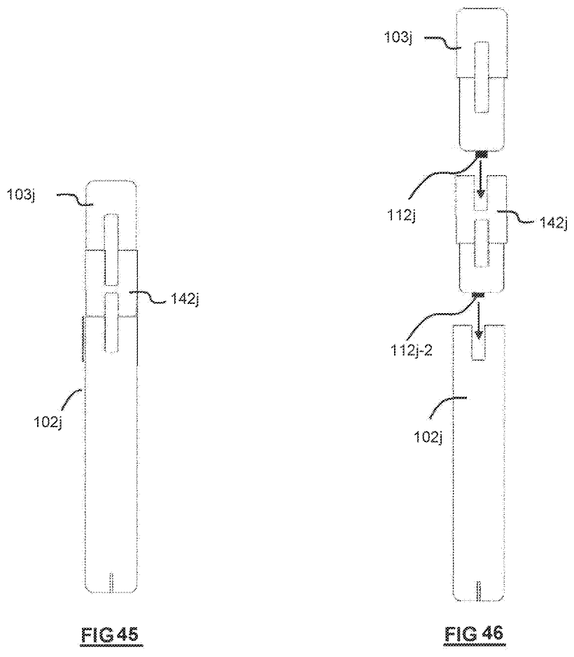

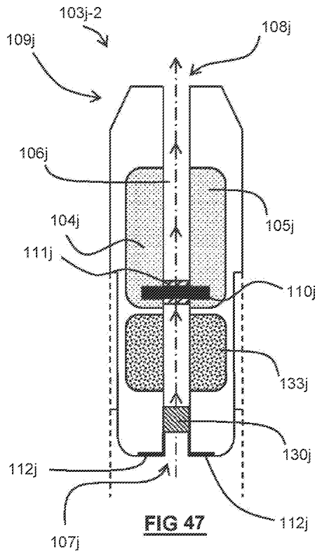

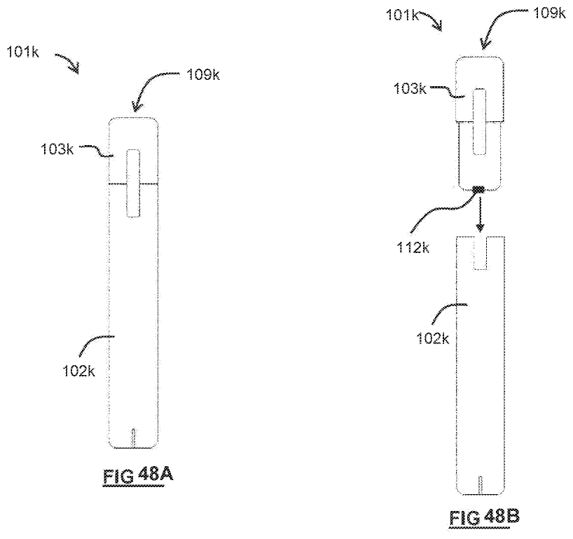

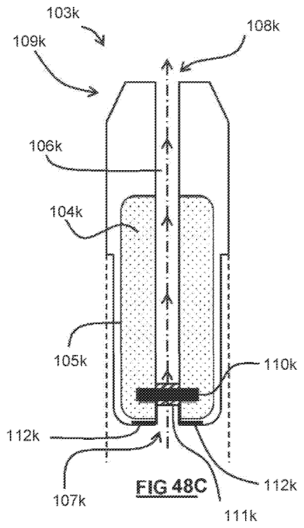

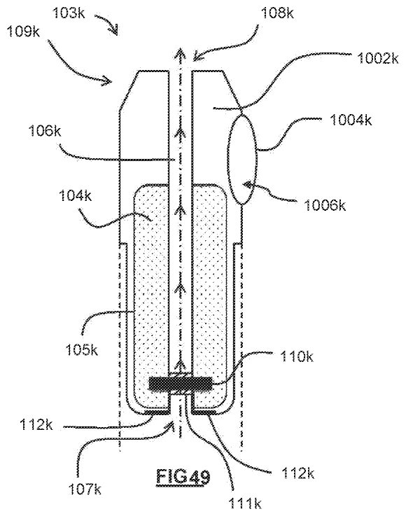

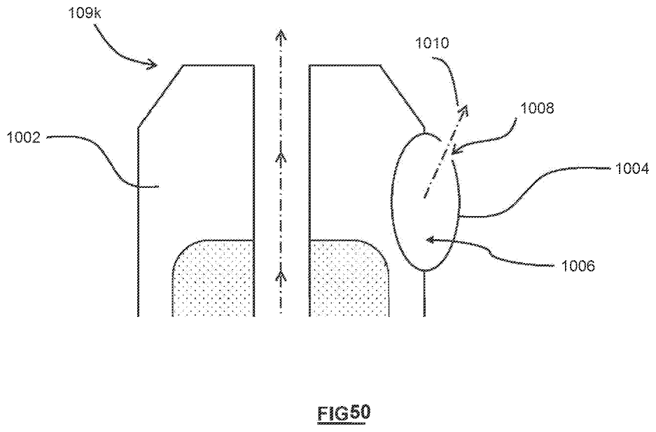

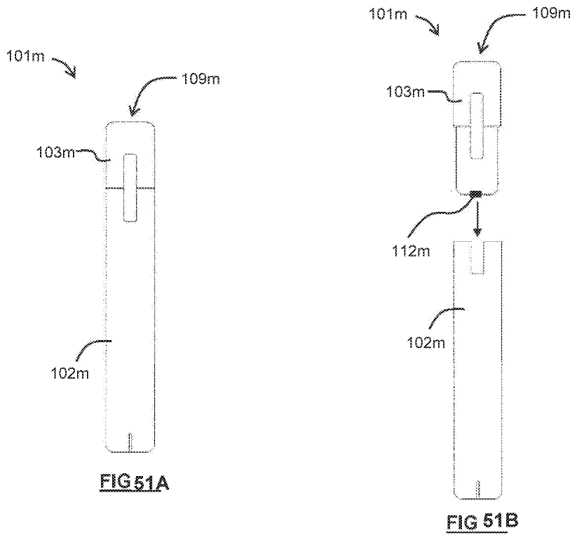

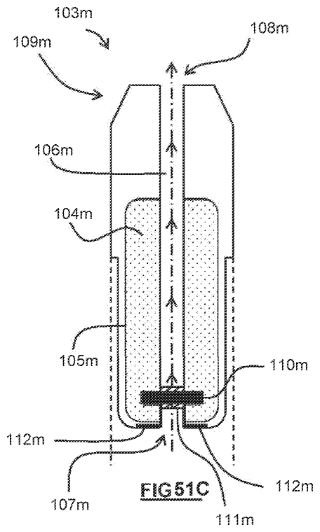

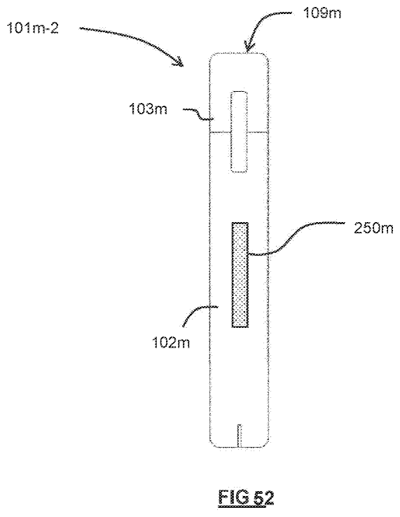

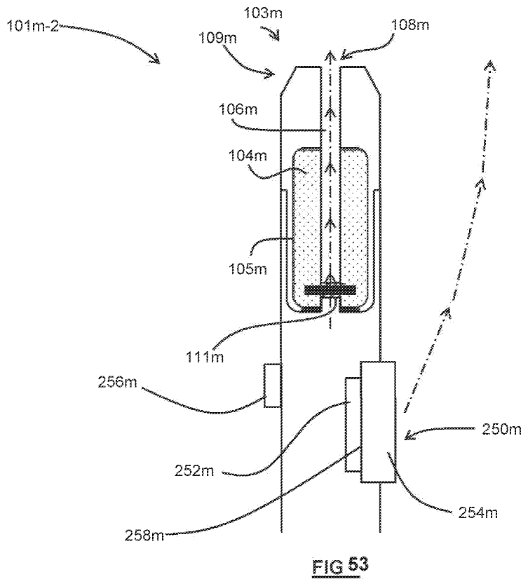

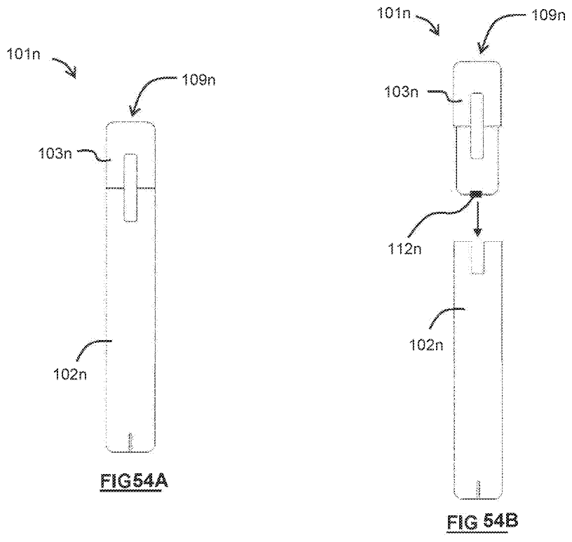

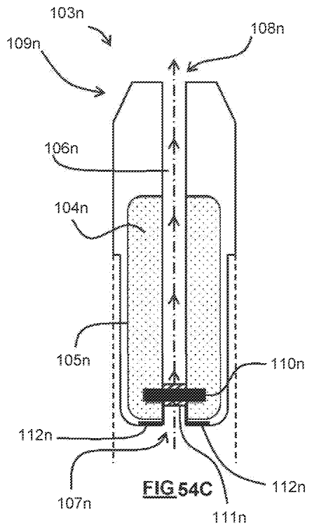

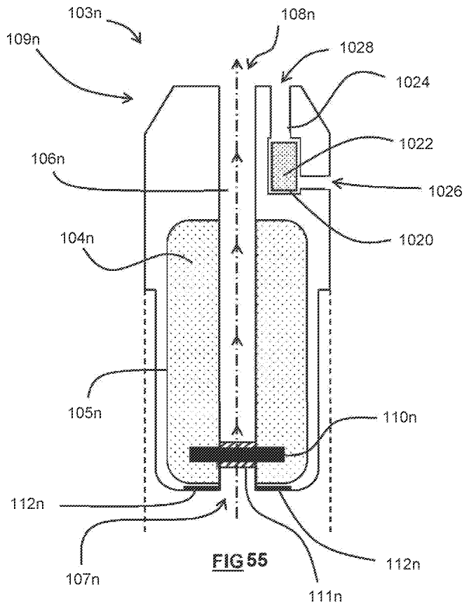

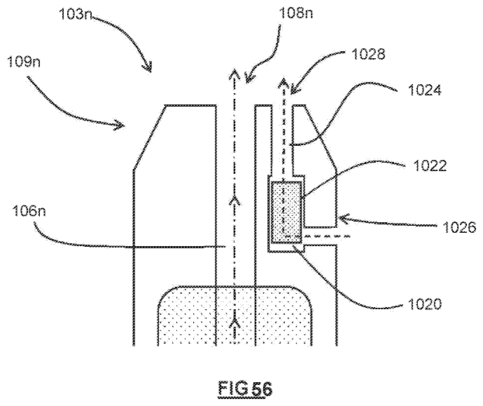

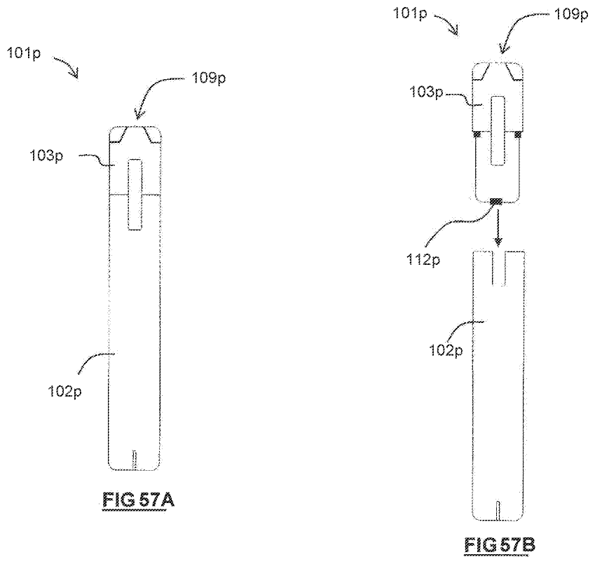

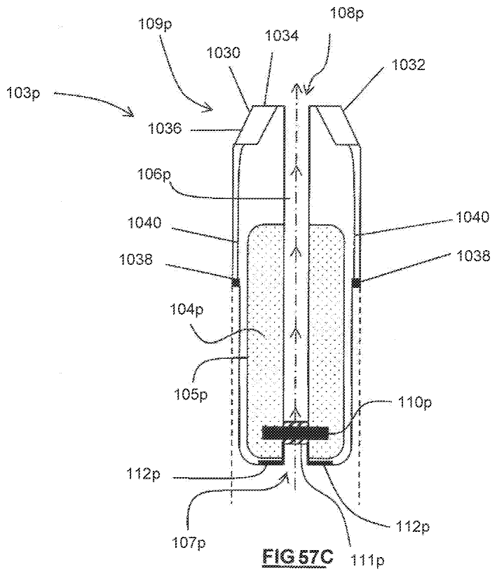

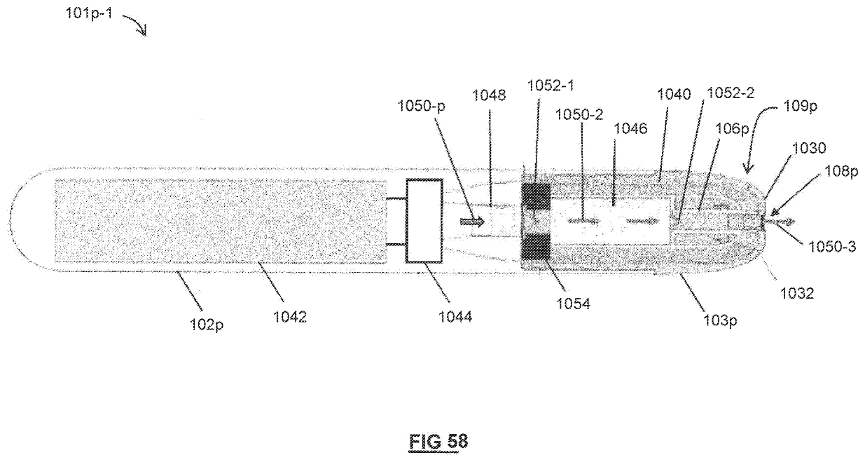

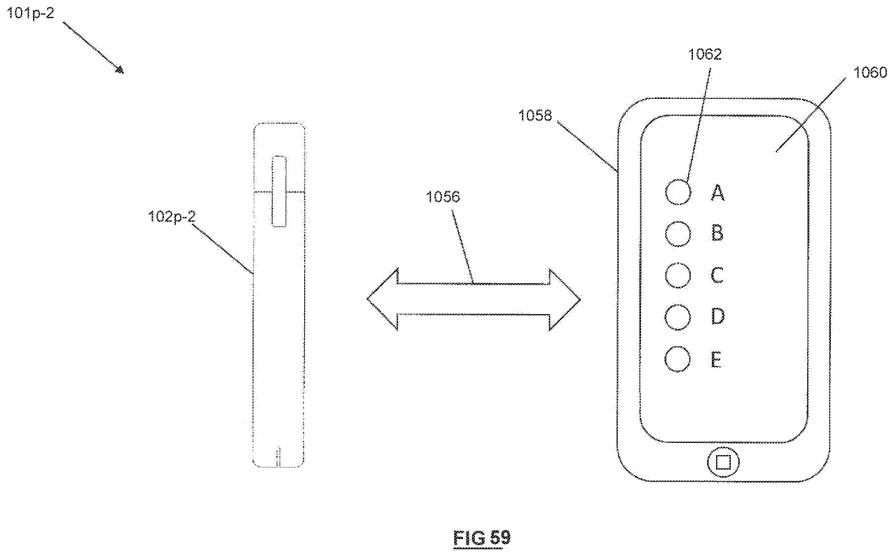

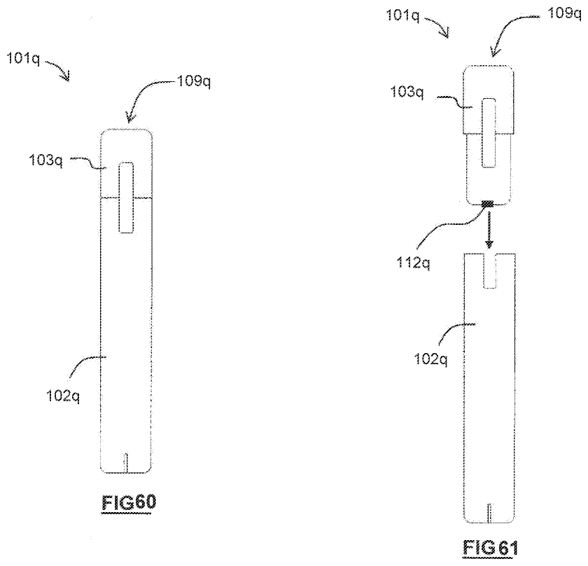

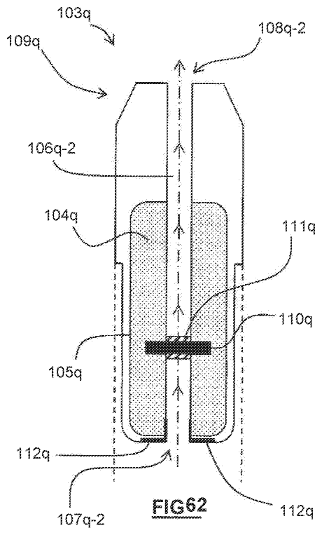

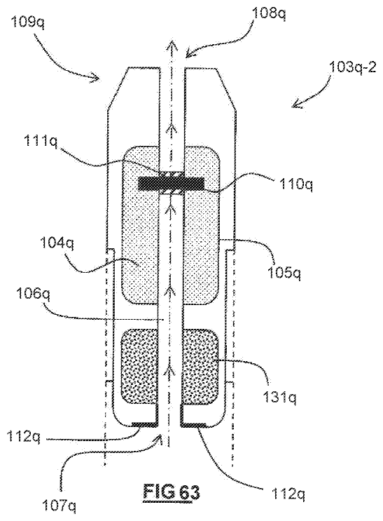

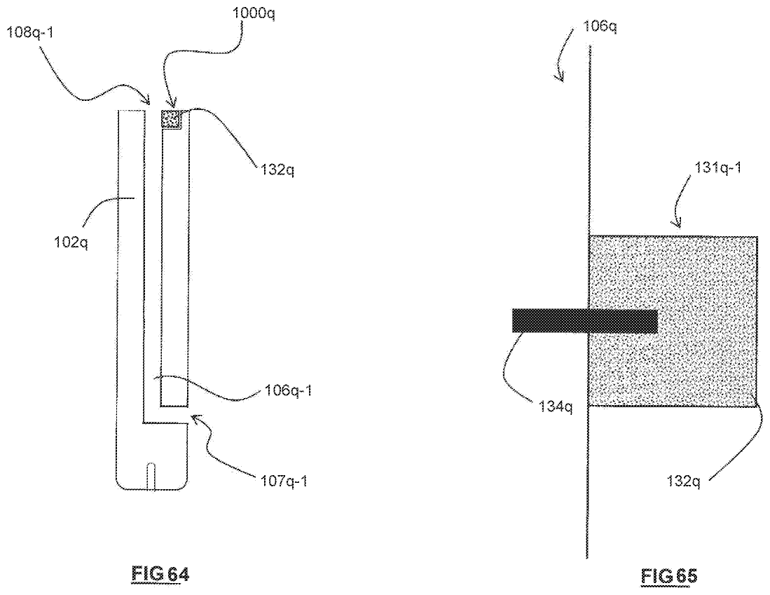

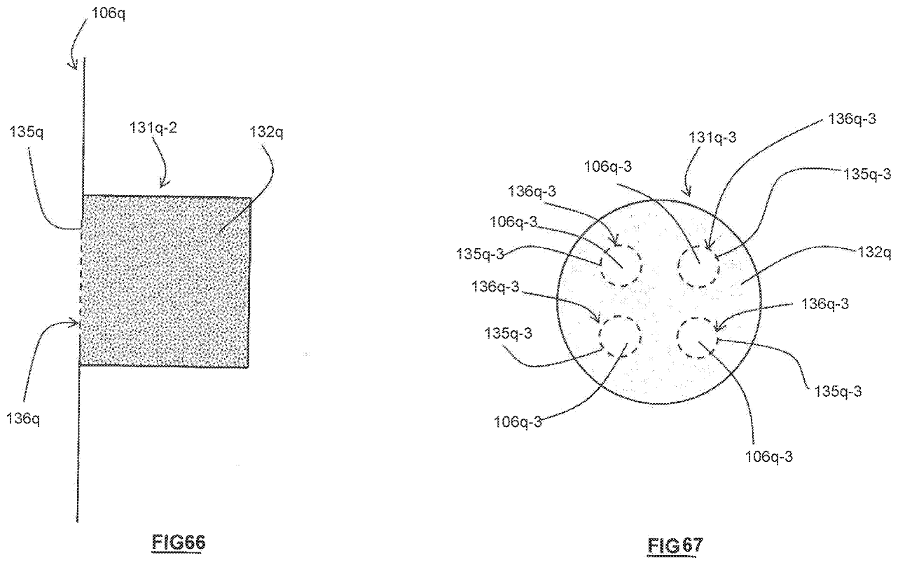

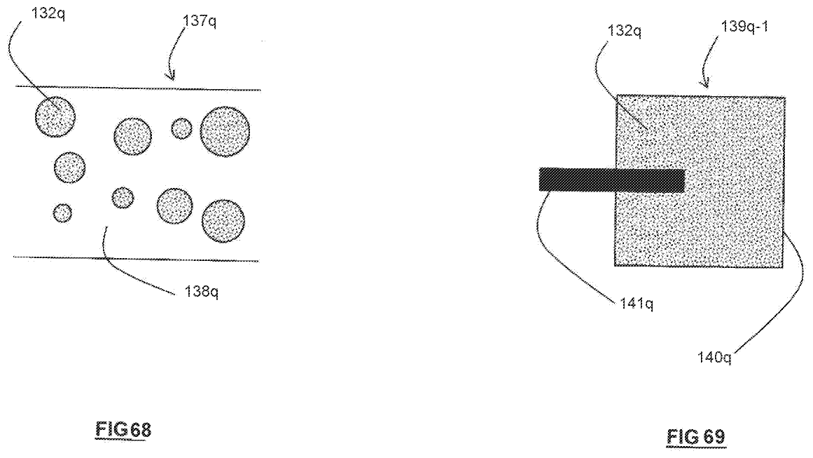

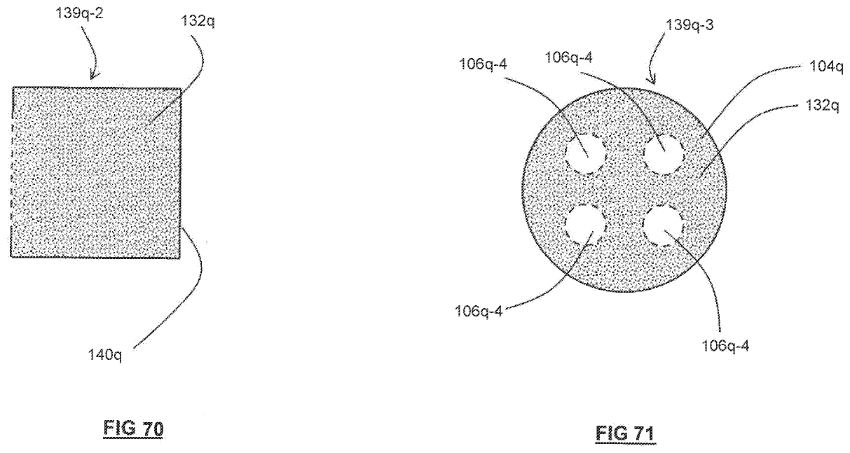

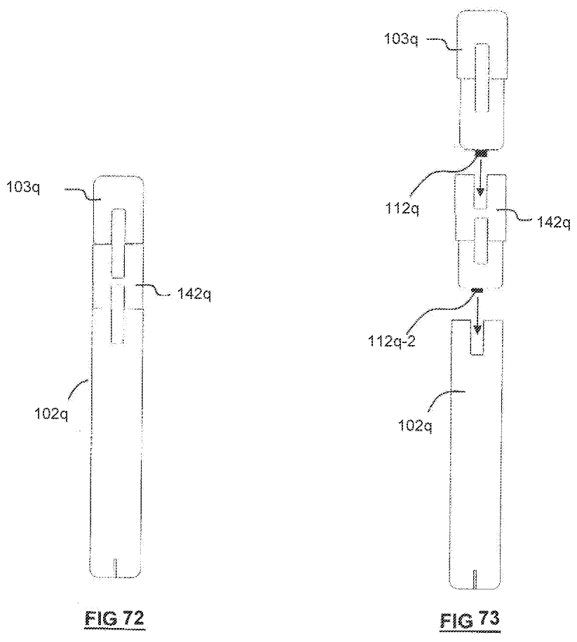

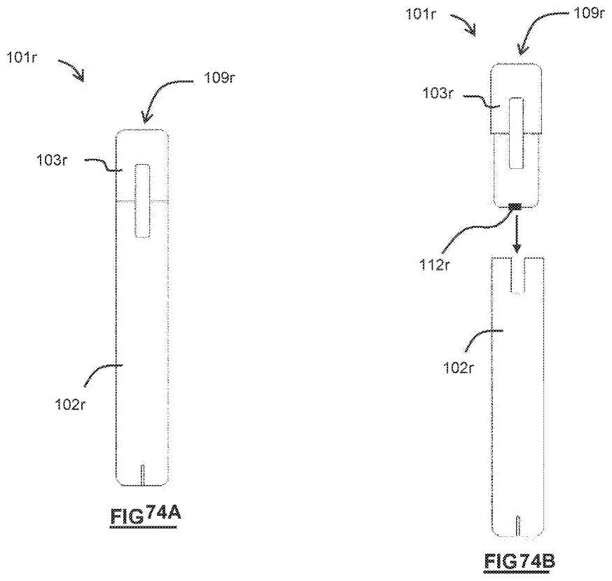

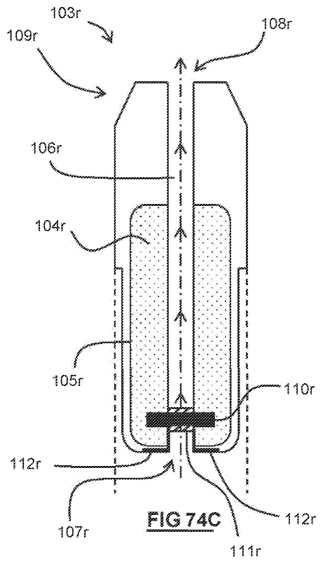

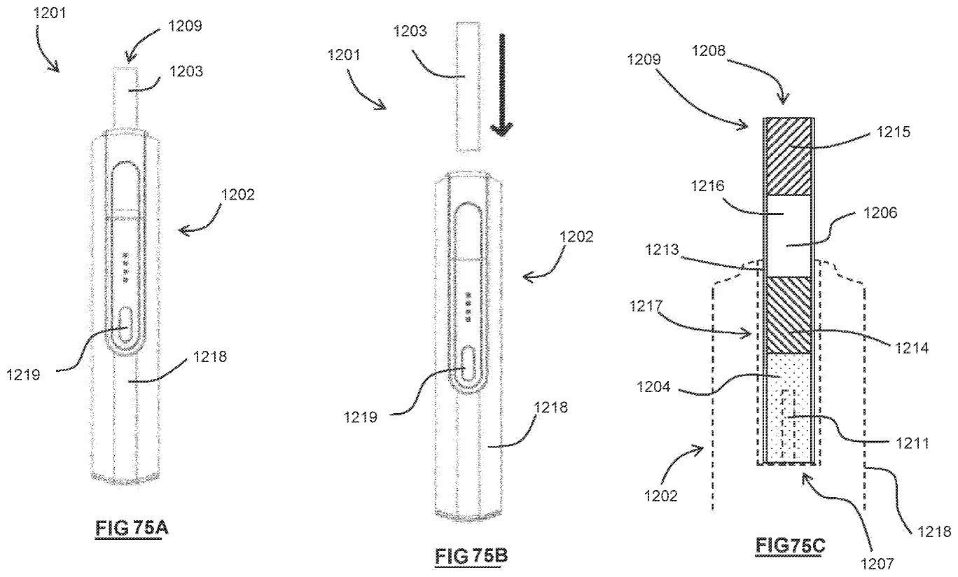

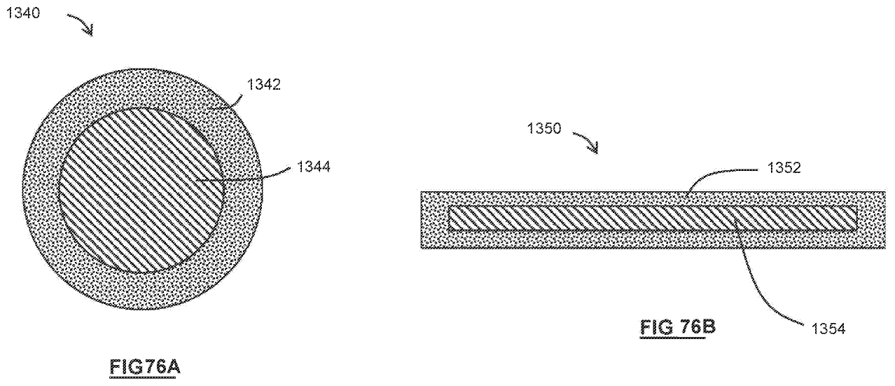

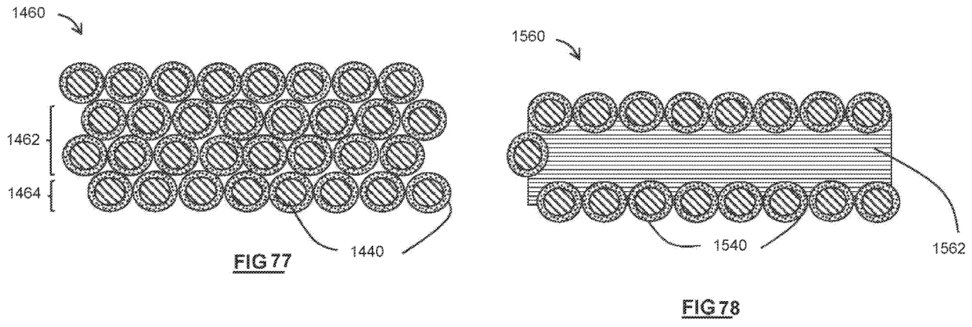

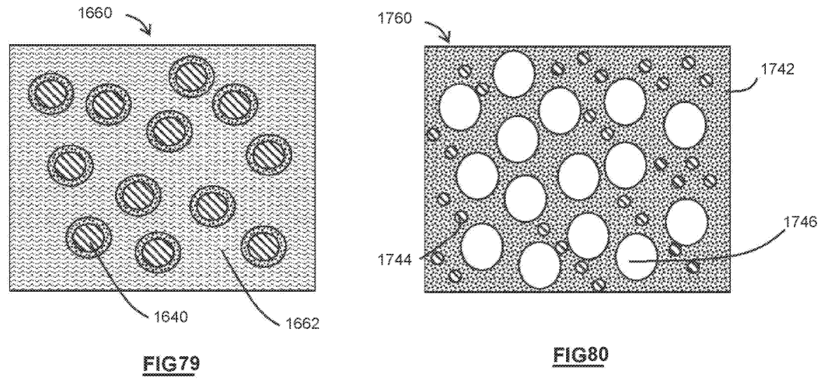

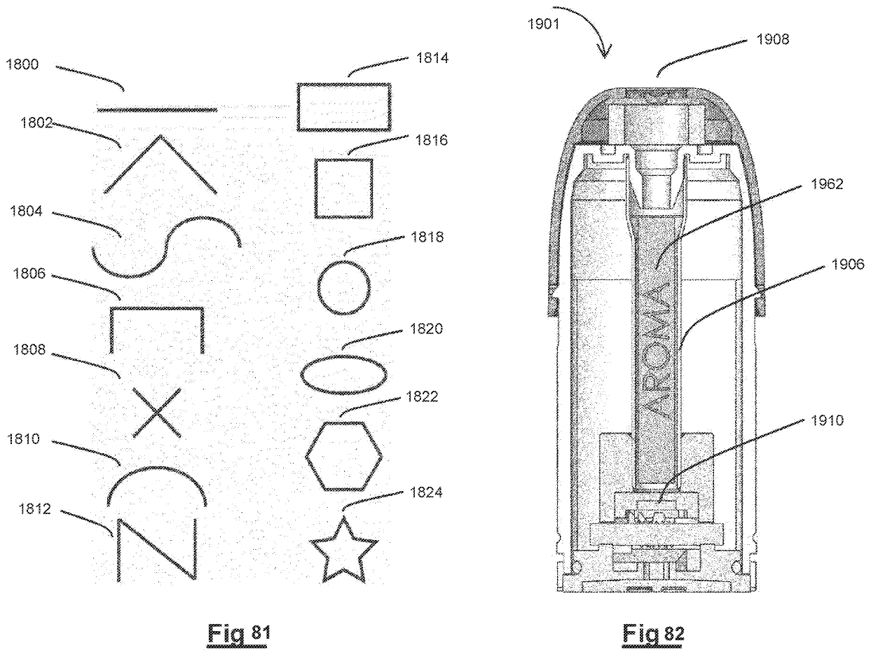

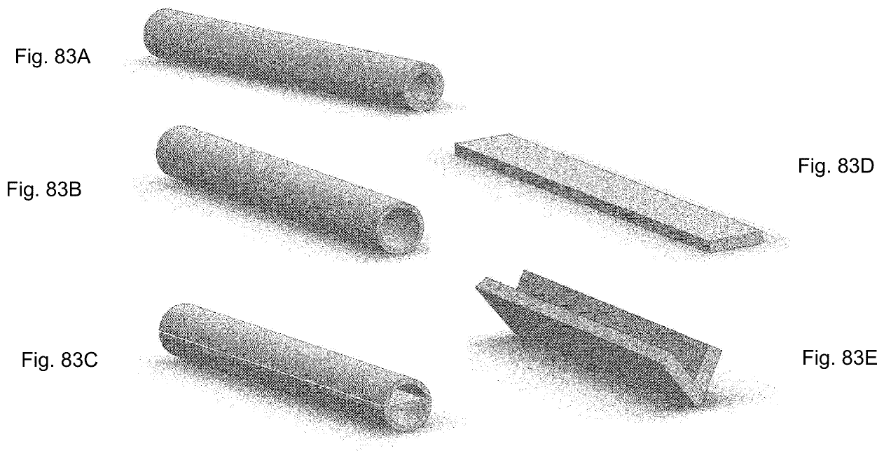

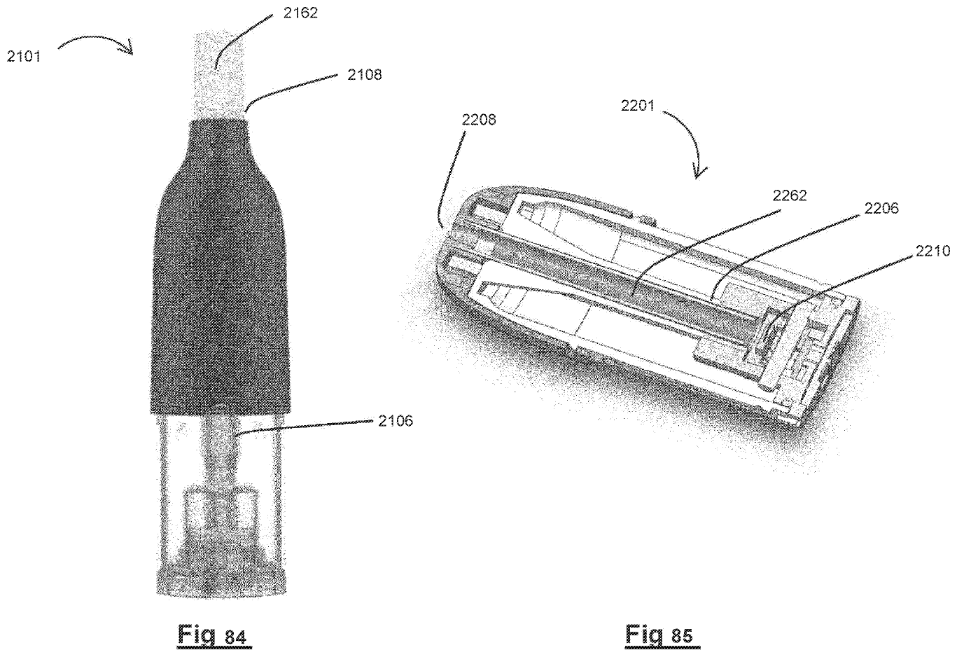

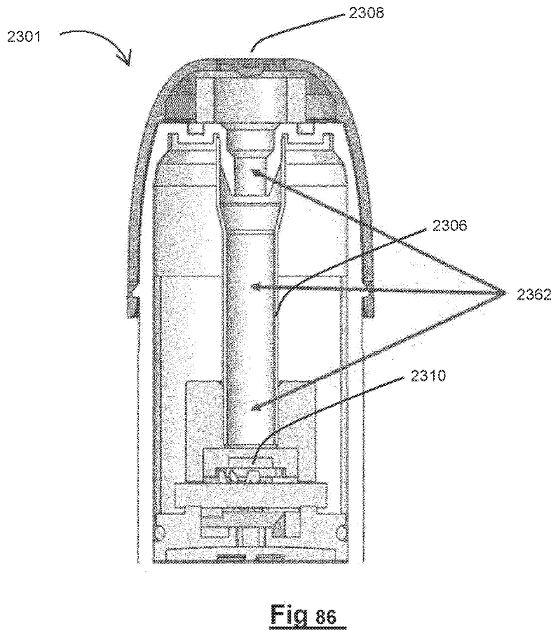

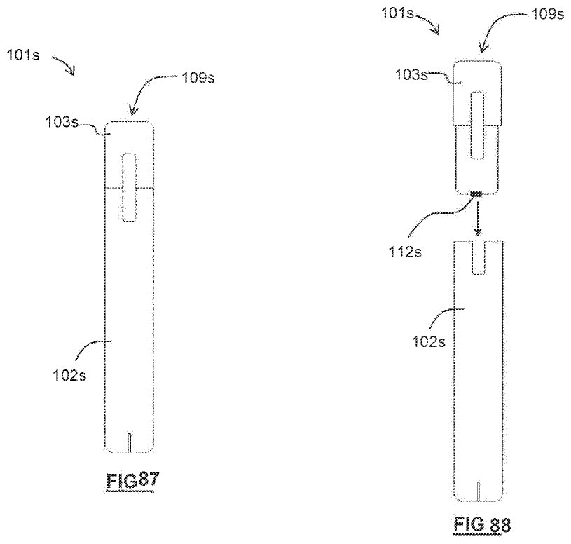

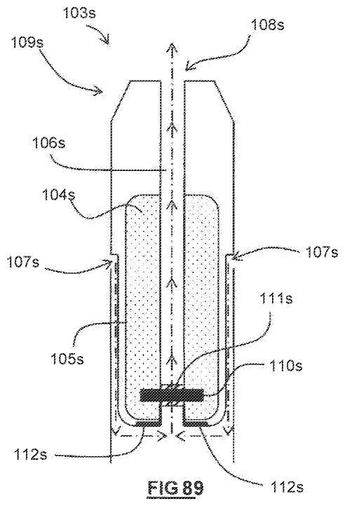

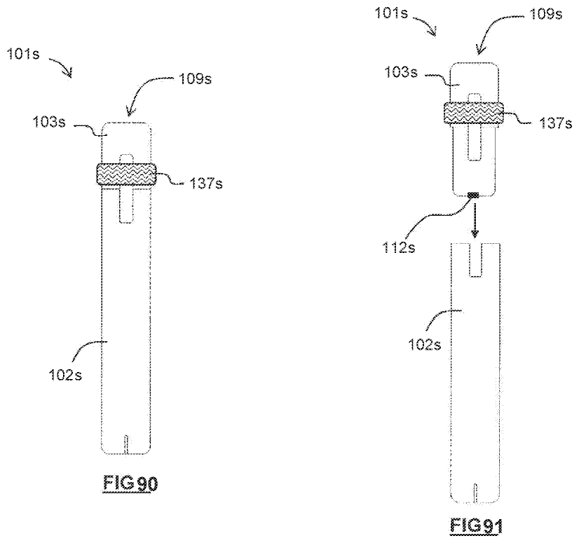

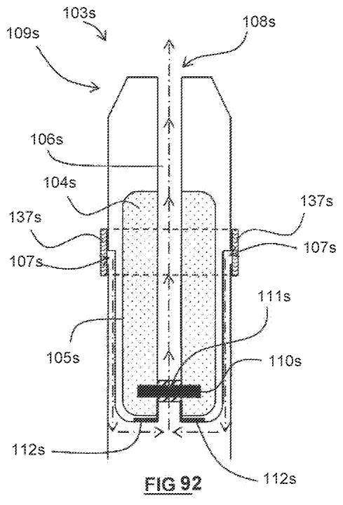

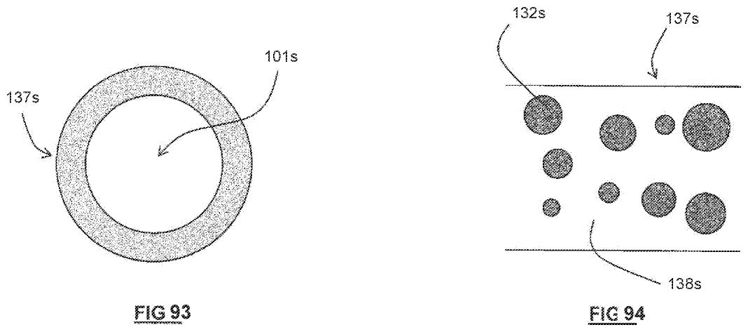

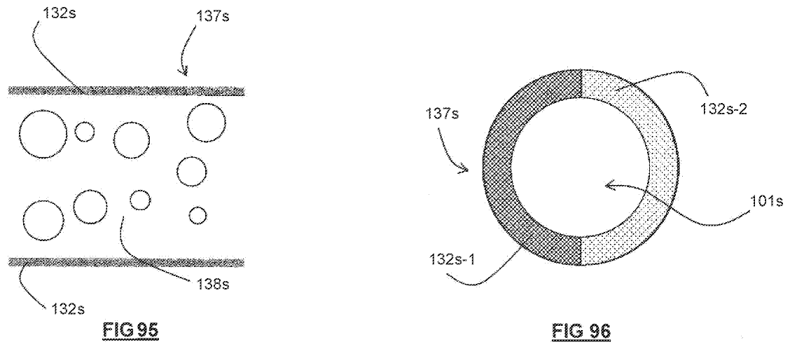

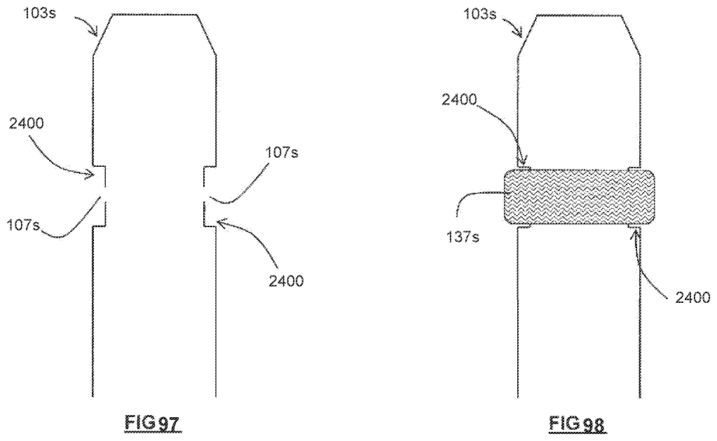

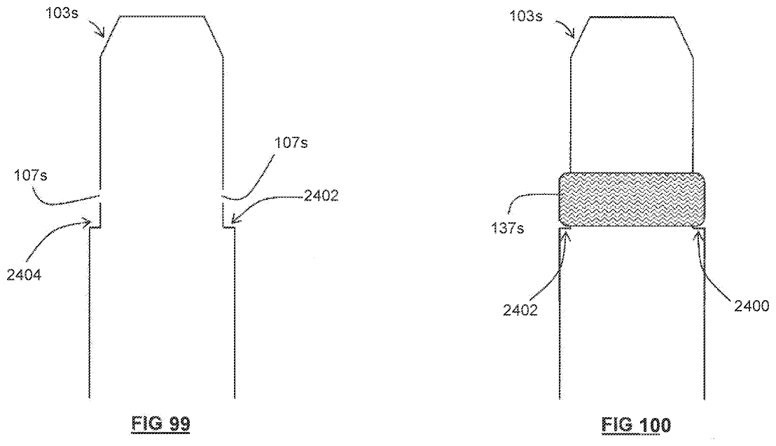

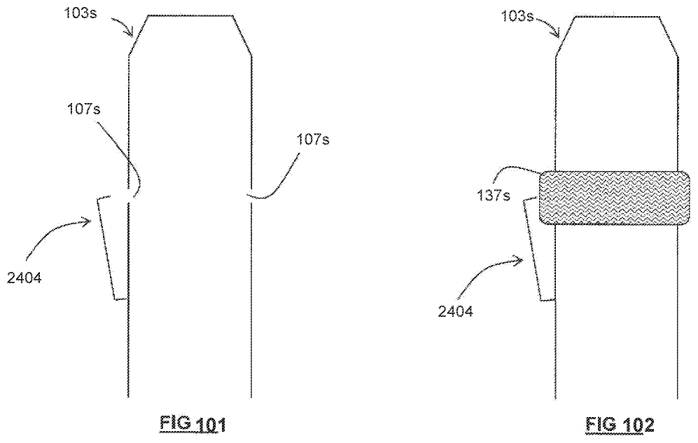

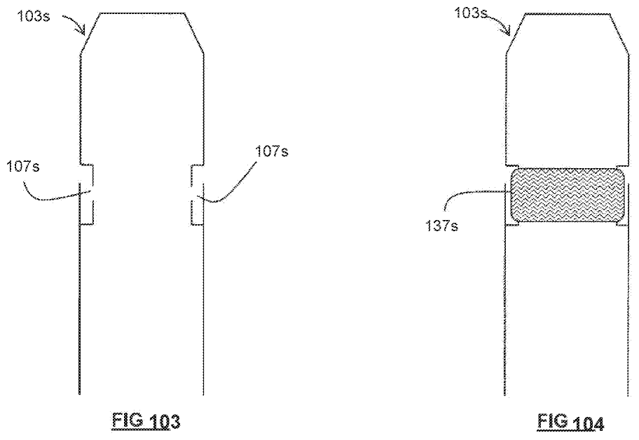

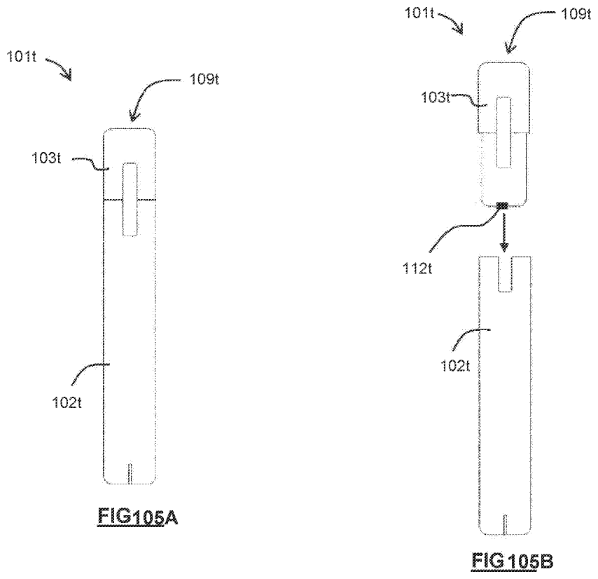

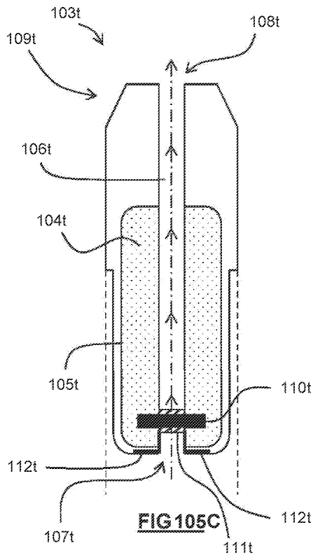

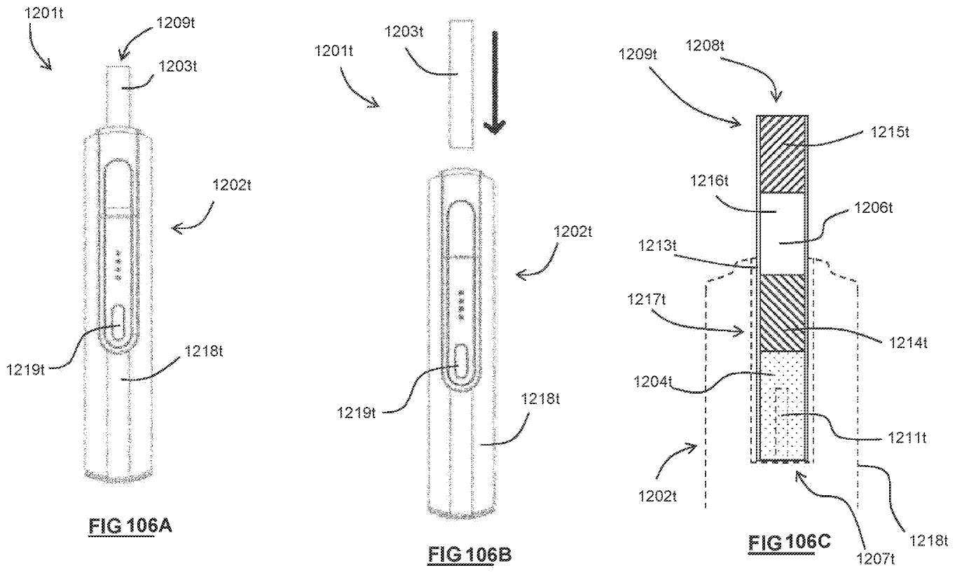

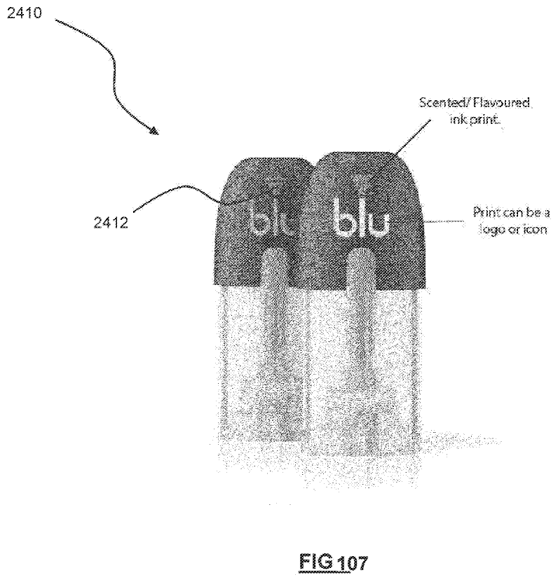

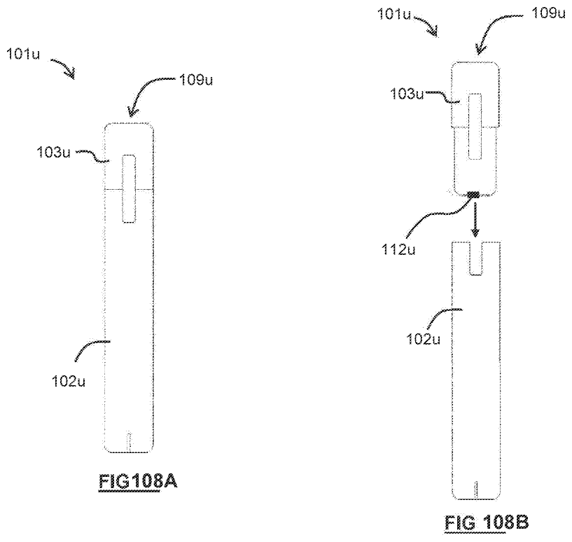

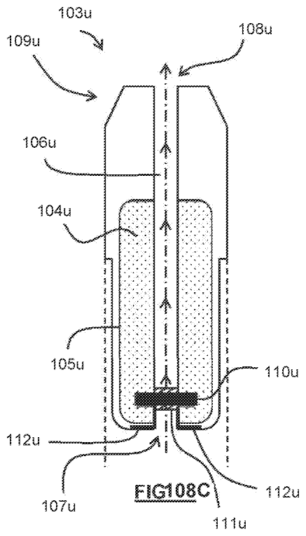

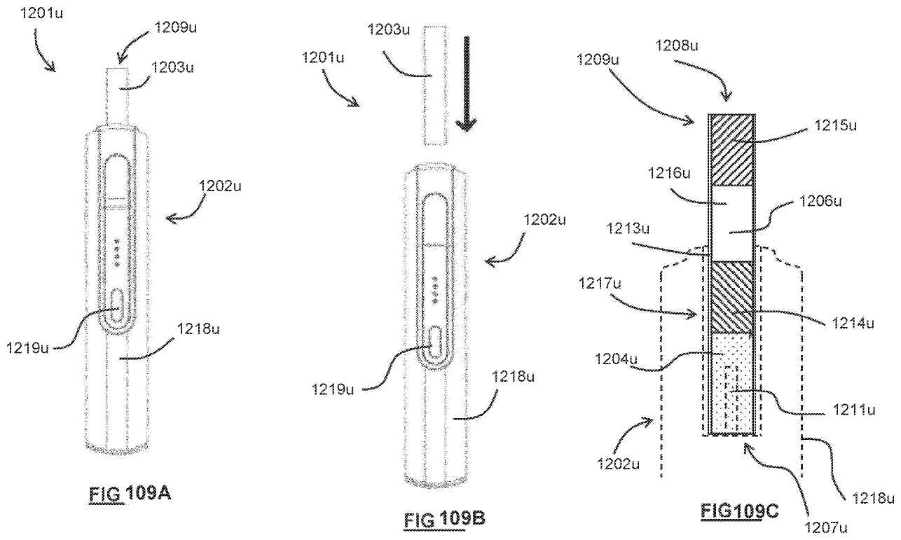

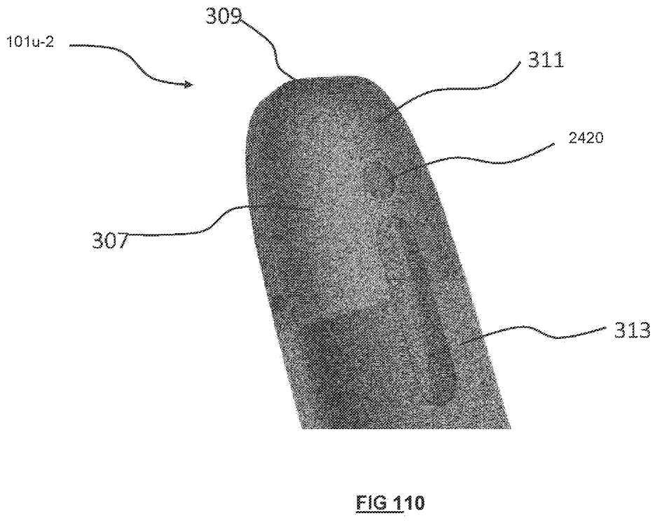

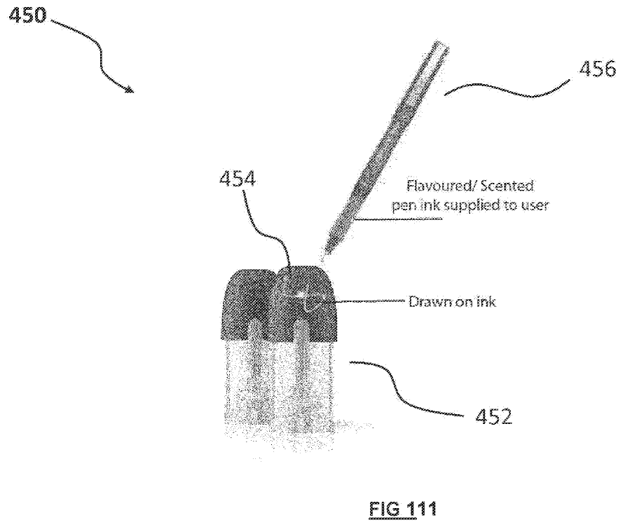

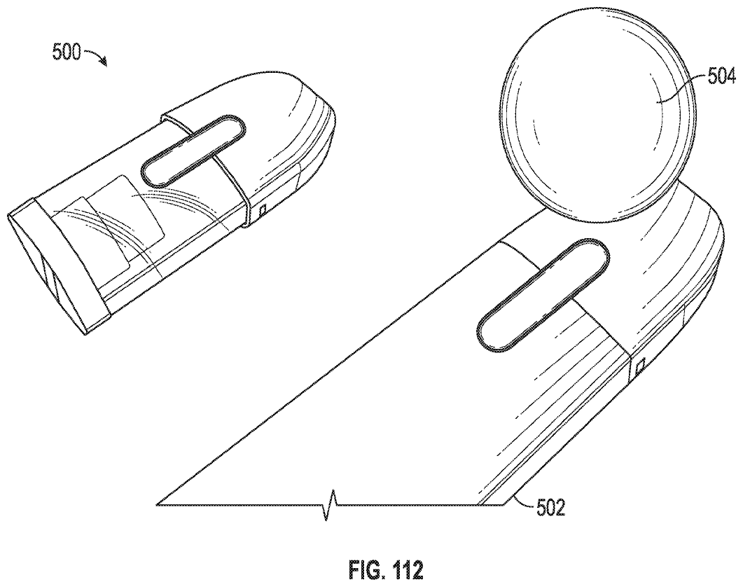

FIG. 3 shows an example of a consumable 400, which on which is printed a visual graphic 403, in this case a logo. The visual graphic 403 is printed using flavored or scented ink, in line with either the first or second aspects of the present invention.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the disclosure in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1-276. (canceled)

278. A smoking substitute apparatus having:
a flavorant-containing element formed by printing a flavorant onto an outer surface of the smoking substitute apparatus.

279. A smoking substitute apparatus according to claim 278, wherein the flavorant-containing element is colorless.

280. A smoking substitute apparatus according to claim 278, wherein the flavorant-containing element comprises a flavored and/or scented ink.

281. A smoking substitute apparatus according to claim 280, wherein the scented and/or flavored substance is printed using pad printing or tampo printing.

282. A smoking substitute apparatus having a flavorant-containing element on an outer surface thereof, the flavorant-containing element comprising a scented and/or flavored ink.

283. A smoking substitute apparatus according to claim 282, wherein the flavorant-containing element includes a scented element and is located on a top surface of the smoking substitute apparatus.

284. A smoking substitute apparatus according to claim 282, wherein the flavorant-containing element includes a flavored element and is located on a bottom surface of the smoking substitute apparatus.

285. A smoking substitute apparatus according to claim 282, wherein the flavorant-containing element is scented and flavored.

286. A smoking substitute apparatus according to claim 282, wherein the smoking substitute apparatus includes a plurality of flavorant-containing elements.

287. A smoking substitute apparatus according to claim 282, wherein the flavorant-containing element is in the form of a visual graphic.

288. A smoking substitute apparatus according to claim 287, wherein the flavorant-containing element is configured to indicate visually how much flavorant is remaining in the flavorant-containing element.

289. A smoking substitute apparatus according to claim 288, wherein the flavorant-containing element is configured to fade over time, and the extent to which the flavorant-containing element has faded provides a visual indication of the amount of flavoring remaining in the flavorant-containing element.

290. A smoking substitute apparatus according to claim 282, wherein the flavorant-containing element includes a first flavorant layer and a second flavorant layer.

291. (canceled)

292-312. (canceled)

313. A smoking substitute apparatus according to claim 278, wherein:
the flavourant-containing element is scented and flavoured.

314. A smoking substitute apparatus according to claim 278, wherein:
the smoking substitute apparatus includes a plurality of flavourant-containing elements.

315. A smoking substitute apparatus according to claims 278, wherein:
the flavourant-containing element is in the form of a visual graphic.

316. A smoking substitute apparatus according to claim 315, wherein:
the flavourant-containing element is configured to indicate visually how much flavourant is remaining in the flavourant-containing element.

317. A smoking substitute apparatus according to claim 316, wherein:
the flavourant-containing element is configured to fade over time, and
the extent to which the flavourant-containing element has faded provides a visual indication of the amount of flavouring remaining in the flavourant-containing element.

318. A smoking substitute apparatus according to claim 278, wherein the flavourant-containing element includes a first flavourant layer and a second flavourant layer.

319. A smoking substitute apparatus according to claim 318, wherein the first flavourant layer has a first flavour and the second flavourant layer has a second flavour.
